SEQUENCE LISTING (9 sequences)

SEQ ID NO: 1 - RNA, 19 nt, Retrovirus
cuagaaaucuguugcuaug

SEQ ID NO: 2 - DNA, 62 bp, Artificial sequence
  synthetic DNA
gatccgctagaaatctgttgctatgttcaagagacatagcaacagatttctagcttttttat

SEQ ID NO: 3 - DNA, 62 bp, Artificial sequence
  synthetic DNA
gatccgtaaggctatgaagagatacttcaagagagtatctcttcatagccttacttttttat

SEQ ID NO: 4 - DNA, 65 bp, Artificial sequence
  synthetic DNA
attgggtaaaggagagtatttgagcggaggacagtactccgggtccccccccccccccccccccc

SEQ ID NO: 5 - DNA, 45 bp, Artificial sequence
  synthetic DNA
gacccggagtactgtcctccgctcttttactctcctttacccaat

SEQ ID NO: 6 - DNA, 3273 bp, Homo sapiens
  CDS (195)..(2294)
cttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctggttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcgggtggctaaggcaggagaatcacttcagcccgggaggtggaggttgcagtgagccaagatcataccacggcactccagcctgggtgacagtgagactgtggctcaaaaaaaaaaaaaaaaaaa

SEQ ID NO: 7 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 8 - DNA, 2530 bp, Homo sapiens
  CDS (74)..(2407)
cagcttccctgtggtttcccgaggcttccttgcttcccgctctgcgaggagcctttcatccgaaggcgggacgatgccggataatcggcagccgaggaaccggcagccgaggatccgctccgggaacgagcctcgttccgcgcccgccatggaaccggatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaaccgtgccctcagaaaatataccattgcctgaatgttcttcaccaccttcatgcaaacgtaaagttggtggtacatcagggaggaaaaacagtaacatgtccgatgaattcattagtctttcaccaggtacaccaccttctacattaagtagttcaagttacaggcaagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacgtggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccagtcattgtactgtttttaatgttcacatttttacaaataggtagagtcattcatatttgtctttgaatc

SEQ ID NO: 9 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRQVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

(19) United States
(12) Patent Application Publication
(10) Pub. No.: US 2009/0130760 A1
(43) Pub. Date: May 21, 2009

(54) METHOD FOR THE UBIQUITINATION OF COMMON SUBUNIT OF RNA POLYMERASES

(75) Inventors: Tomohiko Ohta, Tokyo (JP); Wenwen Wu, Tokyo (JP)

Correspondence Address: DICKSTEIN SHAPIRO LLP, 1177 AVENUE OF THE AMERICAS (6TH AVENUE), NEW YORK, NY, 10036-2714, US

(73) Assignee: ST. MARIANNA UNIVERSITY SCHOOL OF MEDICINE, Kawasaki-shi (JP)

(21) Appl. No.: 12/090,606

(22) PCT Filed: Oct. 18, 2006
(86) PCT No.: PCT/JP2006/321199

§ 371 Date: Oct. 27, 2008

Related U.S. Application Data
(60) Provisional application No. 60/727,429, filed on Oct. 18, 2005.

(51) Int. Cl. C12N 9/96; C12N 15/87; C12N 15/12; C07K 2/00

(52) U.S. Cl. 435/455; 435/188; 536/23.5; 530/350

(57) ABSTRACT

The present invention provides a method for ubiquitinating RNA polymerases, comprising bringing the RNA polymerases into contact with BRCA1-BARD1.

### FIELD OF THE INVENTION

The present invention relates to the field of ubiquitination of RNA polymerases, and more particularly to the function of BRCA1 in the regulation of ubiquitination.

### BACKGROUND OF THE INVENTION

The breast and ovarian cancer suppressor protein BRCA1 acts as a hub protein that coordinates a number of cellular pathways to prevent cancer progression. Approximately 80% of the lifetime risk of breast cancer is caused by a germline genetic mutation of this key gene (King et al., 2003). Thus, it is not difficult to imaging that down-regulation of this protein due to other mechanisms could cause sporadic breast cancer (Baldassarre et al., 2003; Catteau and Morris, 2002). Any cell deficient in BRCA1 shows genomic instability as evidenced by hypersensitivity to DNA damage, the presence of chromosomal abnormalities and the loss of heterozygosity at multiple loci (Al-Wahiby and Slijepcevic, 2005; Xu et al., 1999). These results are likely to have stemmed from deficiency in BRCA1 that plays a role in DNA damage repair, transcriptional control, apoptosis induction, intra-S- or G2-M-phase checkpoint function and centrosome duplication control (Deng, 2002; Ohta and Fukuda, 2004; Venkitaraman, 2002; Zheng et al., 2000). Elucidation of complicated cell mechanisms mediated by BRCA1 is an essential step for understanding occurrence of breast and ovarian cancers, in other words, for developing an effective treatment approach for these cancers.

Involvement of BRCA1 in multiple cellular pathways is theoretically indicated by its enzymatic function as a ubiquitin ligase (E3). With respect to this enzymatic function, BRCA1 possibly interacts with multiple protein substrates and possibly influences biological response of a cell in many respects. BRCA1 contains an N-terminal RING finger domain, a common motif found in ubiquitin ligases.

BRCA1 acquires significant ubiquitin ligase activity when it is bound to another conformationally similar RING finer protein BARD1 as a RING heterodimer (Baer and Ludwig, 2002; Brzovic et al., 2003; Chen et al., 2002; Hashizume et al., 2001; Mallery et al., 2002). Ubiquitin ligase catalyzes the formation of polyubiquitin chain bound to a substrate protein via an isopeptide bond using ubiquitin molecules sequentially activated by enzymes E1 and E2 (Hershko and Ciechanover, 1998). The most common polyubiquitin chain is linked to Lys48 of ubiquitin and serves as a signal for rapid degradation of the substrate via a proteasome-dependent proteolytic pathway (Chau et al., 1989). BRCA1-BARD1, however, has a characteristic function of catalyzing the generation of Lys6-linked polyubiquitin chains (Morris and Solomon, 2004; Nishikawa et al., 2004; Wu-Baer et al., 2003). These chains are recognized by 26S proteasome in vitro not for degradation but for deubiquitinaiton in a ubiquitin aldehyde-sensitive manner (Nishikawa et al., 2004). The substrate of Lys6-linked ubiquitination of BRCA1 including autoubiquitinated BRCA1 has been shown to stay stable in vivo without being degraded (Hashizume et al., 2001; Sato et al., 2004). These findings suggest the possibility of ubiquitination via BRCA1-BARD1 becoming a signal for a process other than degradation.

A harmful missense mutation in the RING finger domain of BRCA1 found in familial breast cancer abolishes E3 ligase activity of BRCA-BARD1 (Brzovic et al., 2003; Hashizume et al., 2001; Ruffner et al., 2001). This shows that E3 ligase activity is important for BRCA1 to play a role as a tumor suppressor protein.

One of the most important functional features of BRCA1 is that it is a component of RNA polymerase II (pol II) holoenzyme (Chiba and Parvin, 2002; Scully et al., 1997a). In an undamaged cell, BRCA1 specifically interacts with majority of hyperphosphorylated processive pol II (IIO) rather than with hypophosphorylated pol II (IIA) found in promoters (Krum et al., 2003). Once DNA damage is caused, BRCA1 is phosphorylated by an ATM/ATR family kinase (Cortez et al., 1999; Tibbetts et al., 2000), and dissociated from the processive pol II complex (Krum et al., 2003). Subsequently, phosphorylated BRCA1 coorporates with Rad50-Mre11-Nbs1 repair complex, Rad51 or PCNA and repairs the damaged DNA (Scully et al., 1997b; Zhong et al., 1999).

BRCA1 has been proposed to bind to a pol IIO complex as part of the genome scanning function (Lane, 2004). The extent of influence of BRCA1 on the pol II complex, if at all, during the early stage after the DNA damage and before BRCA1 translocates to the repair machinery remains to be elucidated.

### DISCLOSURE OF THE INVENTION

The objectives of the present invention are to provide a method for ubiquitinating a common subunit of RNA polymerases, a method for controlling the ubiquitination and a method for establishing a cell sensitive to DNA damage.

In order to solve the above problems, we have gone through keen study and found that a heterodimer (BRCA1-BARD1) consisting of a RING finger protein BRCA1 and another RING finger protein BARD 1 ubiquitinates subunits common in various RNA polymerases. Moreover, we found that mutation of an amino acid sequence of a RNA polymerase subunit could suppress such ubiquitination, and further that such suppression of ubiquitination could impart sensitivity to a DNA-damaging environment caused by ultraviolet (UV) or the like. The present invention was completed based on these findings. Thus, the present invention is as follows.

(1) A method for ubiquitinating a common subunit of RNA polymerases comprising bringing the RNA polymerase into contact with BRCA 1-BARD 1.

An example of the common subunit of RNA polymerases includes RPB8.

(2) A method for suppressing ubiquitination of RPB8 by BRCA1, comprising mutating a lysine residue in the amino acid sequence of RPB8.

(3) A method for producing a cell sensitive to a DNA-damaging environment comprising suppressing ubiquitination of RPB8 through mutation of the amino acid sequence of RPB8 in a cell to impair sensitivity to a DNA-damaging environment to the cell.

(4) A method for treating cancer comprising bringing an RNA polymerase into contact with BRCA1-BARD 1.

This cancer treatment takes an advantage of ubiquitination of the common subunit of RNA polymerases to impair an anticancer activity to the body.

(5) A pharmaceutical composition comprising BRCA1-BARD1.

(6) A pharmaceutical composition comprising a gene coding for BRCA1 and a gene coding for BARD 1.

The pharmaceutical composition of the invention is used for treating cancer.

### BRIEF DESCRIPTION OF THE DRAWINGS

FIG. 1 is a view showing the results of screening for proteins that are affected by epirubicin treatment.

T47D cells (Panels A and B) and HCC1937 cells (Panels C and D) were either untreated or treated with 0.2 μg/ml epirubicin for 3 hours and each lysed with 7M urea/2M thiourea-containing buffer. Proteins (50 μg) obtained from untreated and Epirubicin-treated cells were labeled with fluorescent dyes Cy3 (Panels A and C) and Cy5 (Panels B and D), respectively. The labeled samples were mixed together, separated on a 2D gel (pH range from left to right being 3-10) and scanned with a fluorescence image analyzer. The yellow arrows indicate the spots of the proteins whose level altered widely according to the epirubicin treatment. The red arrows indicate proteins that significantly decreased only in T47D cells by epirubicin treatment. Slower-migrating proteins and faster-migrating proteins were identified as RPB8 and myosin light chain, respectively.

FIG. 2 is a view showing results from immunoblotting of RPB8 modification following epirubicin treatment.

A: T47D cells or HCC1937 cells were untreated (control) or treated with 0.2 μg/ml epirubicin for 3 hours and each lysed with 7M urea/2M thiourea-containing buffer. Lysates (500 μg) were separated on a 2D gel (pH range 3-10). A part of the gel was subjected to immunoblotting with anti-RPB antibody. The arrow indicates RPB8.

B: T47D cells or HCC1937 cells were treated with 0.2 μg/ml epirubicin for the indicated times, lysed with 0.5% NP-40-containing buffer, separated by 12.5% SDS-PAGE and then subjected to immunoblotting with anti-RPB8 antibody or anti-tubulin antibody.

FIG. 3 is a view showing results from immunoblotting for interaction of RPB8 with BRCA1-BARD1.

A: Transfected RPB8 interacts with BRCA1-BARD1. 293T cells were transfected with the indicated plasmids. Whole cell lysates (upper three panels) or immunoprecipitates (lower three panels) were subjected to immunoblotting with the indicated antibodies. Anti-HA/Myc antibody designates immunoblotting with anti-HA-antibody and subsequent reprobe with anti-Myc antibody.

B: Endogenous RPB8 interacts with BARD1. Cell lysates prepared from the indicated cell lines were immunoprecipitated with anti-RPB 1 antibody, anti-RPB8 antibody or rabbit preimmune serum (Pre) for anti-RPB8 for analysis by immunoblotting with the indicated antibodies.

C: In vitro interaction between RPB8 and BARD1. His-BARD¹¹⁴⁻¹⁸⁹ (4 μg) was mixed with 4 μg GST or GST-RPB8 and incubated with glutathione beads for 2 hours followed by extensive washing. The proteins bound to the beads were separated by SDS-PAGE and stained with Sypro Ruby.

D: RPB8 interacts with BRCA1 after UV irradiation. MCF10A cells were untreated (Lane 1) or harvested at 5 minutes (Lane 2), 10 minutes (Lane 3), 60 minutes (Lane 4) or 360 minutes (Lane 5) after UV irradiation (35 J/m²). Whole cell lysates (upper two panels) or immunoprecipitates of anti-RPB antibody (lower three panels) were analyzed by immunoblotting with the indicated antibodies. The arrows indicate BRCA1 in modified forms where the arrowhead indicates the normal migration position of BRCA upon straight immunoblotting.

Asterisk: non-specific reaction products.

E: T47D cells were untreated (Lane 1) or harvested at 10 minutes after UV irradiation (35 J/m²) (Lanes 2 and 3). Cell lysates immunoprecipitated with anti-RPB8 antibody (Lanes 1 and 2) or antibody from a preimmunized animal (Lane 3) were analyzed by immunoblotting with the indicated antibodies.

F: T47D cells were transfected either with control siRNA (Lanes 1-3) or siRNA for BRCA1 (Lanes 4-6). The cells were mock treated (Lanes 1 and 4) or harvested at 10 minutes (Lanes 2 and 5) or 60 minutes (Lanes 3 and 6) after UV irradiation (35 J/m²). Whole cell lysates (upper two panels) or immunoprecipitates with anti-RPB8 antibody (lower two panels) were analyzed by immunoblotting with the indicated antibodies.

IP: immunoprecipitates, IB: immunoblotting.

FIG. 4 is a view showing the results from immunoblotting for RPB8 ubiquitination and stabilization in vivo by BRCA1-BARD1.

A: 293T cells transfected with the indicated plasmids were boiled in 1% SDS lysis buffer, diluted to 0.1% SDS and immunoprecipitated with anti-FLAG antibody-cross linked beads. FLAG-RPB8 was eluted with FLAG peptide, separated by 12.5% SDS-PAGE and subjected to immunoblotting with anti-HA antibody.

B: Polyubiquitination of RPB8 was detected in the same manner as in A except that HA-Ub with a single Lys residue was transfected as indicated (Lanes 1-3). A portion of the cell lysate was subjected to immunoblotting with anti-HA antibody to detect total HA-Ub-conjugated proteins in cells as a control for protein expression (Lanes 4-6).

C: 293T cells in p100 plates were transfected with plasmids encoding FLAG-RPB8 (Lanes 1-4, 0.3 μg) and increasing amount of Myc-BRCA1¹⁻⁷⁷² and HA-BARD1 (Lane 2, 2 μg; Lane 3, 4 μg; Lane 4, 7.35 μg). Total amount of plasmid DNA was adjusted to 15 μg per plate by adding empty pcDNA vector. The steady state level of each protein was analyzed using anti-Myc antibody, anti-HA antibody, anti-FLAG antibody or anti-tubulin antibody.

D: 293T cells were transfected with plasmid encoding FLAG-RPB8 (0.2 μg) and either empty pcDNA3 vector (2 μg, upper panel) or Myc-BRCA1¹⁻⁷⁷² and HA-BARD1 plasmids (1 g each, lower panel). The cells were incubated in cyclohexamide (10 μg) and trased for the indicated time. The cell lysates were then immunoblotted with anti-FLAG antibody.

IP: immunoprecipitates, IB: immunoblotting, asterisk: IgG.

FIG. 5 is a view showing results from immunoblotting for BRCA1-dependent polyubiquitination of FPB8 in response to UV irradiation.

A: HeLa cell lines stably expressing wild-type (Lanes 1-3) or 5KR mutants of FLAG-RPB8 (Lanes 4-6) were irradiated with UV (35 J/m²) and harvested at the indicated time after irradiation. Ubiquitinated RPB8 was detected in the same manner as the method shown in FIG. 4A except that anti-ubiquitin antibody was used for immunoblotting (upper panel). The membrane was reprobed with anti-RPB8 antibody (lower panel).

B: HeLa cell lines stably expressing wild-type FLAG-RPB8 were either transfected with control siRNA (Lane 1), transfected with siRNA for BRCA1 (Lane 2), infected with retrovirus expressing control shRNA (Lane 3) or infected with retrovirus expressing shRNA for BRCA1 (Lane 4). Then, cells were irradiated with UV (35 J/m²) and harvested 10 minutes after the irradiation. The cells were boiled in 1% SDS buffer and subjected to immunoblotting with either anti-BRCA1 antibody (upper panel) or anti-tubulin antibody (middle panel) or to detection of RPB8 ubiquitination by the same method as in A (lower panel).

IP: immunoprecipitates, IB: immunoblotting, asterisk: IgG.

FIG. 6 is a view showing results of construction of ubiquitin-resistant RPB8 mutant and assay of its RNA polymerase activity.

A: Mutant constructs of RPB8. Lys (K) residues of RPB8 was substituted with Arg (R) residue as indicated.

B: Myc-BRCA 1¹⁻⁷⁷², BARD1 and HA ubiquitin were co-transfected into 293T cells either with wild-type or mutant FLAG-RPB8. RPB8 polyubiquitination was detected by the same method as that shown in FIG. 4A.

C: 293T cells were transfected with empty pcDNA3 vector (−), wild-type or 5KR mutant of FLAG-RPB8. Whole cell lysates (upper four panels) or anti-FLAG immunoprecipitates from equal amounts of whole cell lysates (lower three panels) were subjected to immunoblotting using the indicated antibodies.

D: The anti-FLAG immunoprecipitates obtained in the same method as that shown in Figure C were subjected to in vitro run-off transcription assay using double-stranded DNA template designed to produce an RNA transcripts of 45 nucleotides. Radiolabelled RNA products were separated with 12% polyacrylamide/urea gel and scanned with a Typhoon 9400®(t image analyzer.

IP: immunoprecipitates, IB: immunoblotting, asterisk: IgG.

FIG. 7 is a view showing that ubiquitin-resistant RPB8 causes UV sensitivity.

A: Cell lysates obtained from 2 HeLa cell clones stably expressing either wild-type (WT-1 and WT2) or 5KR mutant (5KR-1 and 5KR-2) of RPB8, and cell lysates obtained from parent HeLa cells were immunoprecipiated with anti-RPB8 antibodies and subsequently immunoblotted with anti-RPB8 antibody. Arrow: FLAG-RPB8, arrowhead: RPB8.

B: HeLa cell lines in A was irradiated with UV at the indicated doses. Cell viability was determined 48 hours after the irradiation by Trypan Blue exclusion method. The cell number at 0 hours (indicated as 0 J/m²) is 100%. Mean and SD value of the measurements carried out in triplicate are shown. Experiments were repeated at least twice with similar results.

C and D: Cell viability of the indicated HeLa cells previous to (cont., left panel) and 48 hours after (UV, right panel) the UV irradiation at 35 J/m² were observed either by phase contrast microscopy (C) or Lillie's crystal violet staining (D).

FIG. 8 is a view showing that ubiquitin-resistant RPB8 causes prolonged RPB 1 hyperphosphorylation after UV irradiation.

HeLa cells that stably express either wild-type or 5KR mutant of FLAG-RPB8 were irradiated with UV (35 J/m²) and cultured for the indicated time. Whole cell lysates (upper two panels) or anti-FLAG immunoprecipitates from equal amounts of whole cell lysates (lower two panels) were subjected to immunoblotting with the indicated antibodies.

p-S5: Phospho-specific antibody to phosphorylated Ser5 of RPB1.

### BEST MODES FOR CARRYING OUT THE INVENTION

Hereinafter, the present invention will be described in detail.

All patent applications, patents, literatures and websites cited herein are hereby incorporated by reference in their entirety.

We indicated that BRCA1 ubiquitinates subunit RPB8 (also referred to as hRPB17 or POLR2H) common to three types of RNA polymerases in response to DNA damage.

RPB8 was identified by protein screening as a protein modified in epirubicin-treated BRCA1-positive cells. RPB8 interacts with a BRCA1-BARD1 complex, sensitive to BRCA1 knockdown by RNAi and polyubiquitinated immediately after UV irradiation. On the other hand, RPB8 retains its polymerase activity and remains unubiquitinated by substitution of its five lysine residues with arginine residues. Interestingly, HeLa cell lines stably expressing this ubiquitin-resistant form of RPB8 is sensitive to UV and prolonged phosphorylation of RNA polymerase II after DNA damage by UV. This feature can be observed as stalled pol II in Cockayne syndrome cells (Rockx et al., 2000; van den Boom et al., 2002). This result suggests a novel mechanism for responding to DNA damage that is mediated by the E3 ligase activity of BRCA1 through a novel substrate RPB8. Ubiquitination of RPB8 by BRCA1 is critically important for proper execution of the transcription-coupled DNA repair pathway.

The present invention provides a method for ubiquitinating a subunit common to RNA polymerases comprising bringing the RNA polymerases into contact with BRCA1-BARD1. The present invention also provides a method for suppressing RPB8 ubiquitination by BRCA1 comprising mutating lysine residues of the amino acid sequence of RPB8.

According to the present invention, the step of bringing the RNA polymerases into contact with BRCA1-BARD1 is not particularly limited, and RNA polymerases can interact with genetically engineered BRCA1-BARD1 obtained from BRCA1-BARD1 expression plasmid. Conditions for the RNA polymerases interaction can appropriately be determined by one skilled in the art. For example, the step may comprise reaction with a suitable buffer at 37° C. for over an hour.

According to the present invention, suppression of RPB8 ubiquitination by mutating the amino acid sequence of RPB8 in cells can impart sensitivity to the cells to a DNA-damaging environment. A method for preparing such a cell imparted with sensitivity to a DNA-damaging environment is also comprised in the present invention.

The types of cells used are not particularly limited, and may include various cells such as normal cells and cancer cells. Preferably, the cells are mammal cells.

Mutation of an amino acid sequence may employ usual site-directed mutagenesis for introducing mutation into DNA encoding the amino acid sequence. For example, mutagenesis kits employing site-directed mutagenesis such as Kunkel method or Gapped duplex method, specifically QuikChange™ Site-Directed Mutagenesis Kit (Stratagene), GeneTailor™ Site-Directed Mutagenesis System (Invitrogen) or TaKaRa Site-Directed Mutagenesis System (Mutan-K, Mutan-Super Express Km, etc.: Takara Bio) may be used.

### 1. DEFINITIONS

BRCA1 is a breast and ovarian tumor suppressor gene that is one of the most crucial genes in the field of breast cancer research. BRCA 1 acquires high ubiquitin ligase activity when it forms a RING heterodimer (complex) with BARD 1. Targeted disruption of mouse Brca1 gene causes excessive centrosome replication and genomic instability. BRCA1 localizes to the centrosomes during mitosis. BRCA1 is also reported to bind to γ-tubulin.

BARD1 was identified as a RING finger protein (BRCA1-associated Ring Domain 1) that binds to BRCA1.

Although BRCA1 and BARD1 forms a complex through binding with each other and constitutes a RING heterodimer ubiquitin ligase, this ligase activity is completely inactivated due to missense mutation in BRCA1 that leads to familial breast cancer.

BRCA 1-BARD 1 controls various cellular processes such as DNA repair, cell-cycle progression and centrosome replication. Immunocytochemical staining experiments gave the behaviors of NPM (nucleophosmin), BRCA1 and BARD1 during the cell cycle. Specifically, NPM is localized in the nucleolus whereas BRCA1 and BARD1 are localized in the nucleus other than the nucleolus during interphase. They are, however, co-localized around the nucleus near the mitotic spindle and in the centrosome (spindle pole) during the mitotic phase. In addition, in a test using HeLa cells arrested in mitotic phase with a thymidine-nocodazole block, NPM was found to be polyubiquitinated in a short time of transition from mitotic phase to G1 phase. For the observation of intracellular localization and co-localization with NPM during the cell cycle, rabbit polyclonal antibody to C terminus of BARD 1 can be prepared and used. Cell cycle synchronization can be monitored by flow cytometry and in vivo NPM ubiquitination can be assessed by IP-western analysis.

Polyubiquitin chains catalyzed by BRCA1-BARD1 is Lys⁻⁶-linked ubiquitin chains instead of conventional Lys⁻⁴⁸-linked ubiquitin chains that serve as a signal for protein degradation by 26S proteasome. These chains are deubiquitinated in vitro by 26S proteasome^{2,3}. NPM is also ubiquitinated and stabilized in vivo with BRCA1-BARD1 but this ubiquitination does not serve as a signal for 26S proteasome-dependent protein degradation.

Here, the nucleotide sequence and the amino acid sequence of gene encoding BRCA1 used with the present invention are represented by SEQ ID NOS: 6 and 7, respectively. The nucleotide sequence and the amino acid sequence of gene encoding BARD1 are represented by SEQ ID NOS: 8 and 9, respectively.

BRCA1 and BARD1 used with the present invention are not limited to the genes having the nucleotide sequences represented by SEQ ID NOS: 6 or 8, but they may consist of a coding region or a part thereof. For example, the coding region of BRCA1 is 195-2294 region among the nucleotide sequence represented by SEQ ID NO: 6 while the coding region of BARD1 is 74-2294 region among the nucleotide sequence represented by SEQ ID NO: 8. Furthermore, there are also genes encoding a protein that hybridizes with a sequence complementary to these nucleotide sequences under stringent conditions and that has RPB8 ubiquitinating activity. “Stringent conditions” refer to conditions where the salt concentration is 100-500 mM, preferably 150-300 mM and the temperature is 50-70° C., preferably 55-65° C. for washing upon hybridization. Moreover, nucleotide sequences having at least 80% or higher, preferably 90% or higher, more preferably 95% or higher, still more preferably 97% or higher identity (homology) with the nucleotide sequences represented by SEQ ID NOS: 6 or 8 can also be used with the present invention.

Proteins having an amino acid sequences varied by deletion, substitution, addition or a combination thereof of one or several amino acids in the amino acid sequence represented by SEQ ID NOS: 7 or 9 and having RPB8-ubiquitinating activity can also be used with the present invention. Examples of such amino acid sequences include: (i) an amino acid sequence having 1-9 (e.g., 1-5, preferably 1-3) amino acids deleted in the amino acid sequence represented by SEQ ID NO: 7 or 9; (ii) an amino acid sequence having 1-9 (e.g., 1-5, preferably 1-3) amino acids substituted with other amino acids in the amino acid sequence represented by SEQ ID NO: 7 or 9; (iii) an amino acid sequence having 1-9 (e.g., 1-5, preferably 1-3) amino acids added to the amino acid sequence represented by SEQ ID NO: 7 or 9; and (iv) an amino acid sequence varied by any combination of (i)-(iii) above. For the substitution of one or several amino acids in the amino acid sequence represented by SEQ ID NO: 7 or 9, the amino acids are preferably substituted with amino acids having similar characteristic with the amino acid to be substituted. Thus, substitution between similar amino acids, for example, between acidic amino acids or between basic amino acids, is favorable as substitution that retains the property of the protein. There is no limitation to the number and the site of the amino acids to be substituted. In addition, a protein having at least 80% or higher, preferably 90% or higher, more preferably 95% or more, still preferably 97% or higher identity (homology) with the amino acid sequence represented by SEQ ID NO: 7 or 9 can also be used with the present invention. A part of these amino sequences is also usable.

DNA encoding an amino acid sequence with variation such as deletion, substitution or addition of one or several amino acids in the amino acid sequence represented by SEQ ID NO: 7 or 9 is prepared according to a method such as a site-directed mutagenesis described in “Molecular Cloning, A Laboratory Manual 2nd ed.” (Cold Spring Harbor Press (1989)), “Current Protocols in Molecular Biology” (John Wiley & Sons (1987-1997)), Kunkel (1985) Proc. Natl. Acad. Sci. USA 82: 488-92, Kunkel (1988) Method. Enzymol. 85: 2763-6.

Introduction of mutation into DNA for the purpose of preparing a protein having such mutation can be carried out with a mutagenesis kit using site-directed mutagenesis such as Kunkel method or Gapped duplex method, for example, QuikChange™ Site-Directed Mutagenesis Kit (Stratagene), GeneTailor™ Site-Directed Mutagenesis System (Invitrogen), TaKaRa Site-DIrected Mutagenesis System (Mutan-K, Mutan-Super Expression Km, etc.: Takara Bio).

BRCA1-BARD can be obtained by expressing each of the genes for producing their proteins and mixing them. BRCA1 and BARD can also be co-expressed. These techniques are well known in the art (see “Molecular Cloning, A Laboratory Manual 2nd ed.” (Cold Spring Harbor Press (1989))).

NPM is a substrate of BRCA1-BARD1 ubiquitin ligase. It has multiple biological activities such as ribosome biosynthesis, apoptosis suppression, histone chaperon action in every cells. An example of nucleophosmin includes nucleophosmin/B23/NO38 (NPM).

Herein, “ubiquitination” (Ub-modification) refers to a rapid and reversible post-translational modification of intracellular proteins with polyubiquitin chains, a process that sequentially binds ubiquitins onto substrate proteins through cooperation of enzymes, i.e., a ubiquitin-activating enzyme (E1), a ubiquitin-conjugating enzyme (E2) and a ubiquitin ligase (E3).

This process initiates with formation of a thiol ester linkage between E1 and the C terminus of Ub, followed by transfer of Ub to the active site Cys of E2. For the most part of this process, formation of isopeptide bonds between the C termini of Ub and lysine residues of the substrates involves a protein or a protein complex known as an E3. E3 recognize E2 and facilitate the transfer of Ub from E2 to the substrate. E3 plays an important role in catalyzing the formation of chains of Ub molecules on substrates that are crucial for recognition by proteasomes.

The 76-residue polypeptide, ubiquitin, fulfils essential functions in eukaryotes through its covalent attachment to other intracellular proteins. The best characterized role for this modification is the targeting of proteins for degradation by 26S proteasome after the transfer of an ubiquitin chain of at least four units, which owes to ubiquitin-ubiquitin linkage at Lys-48 of the ubiquitins. This is referred to as polyubiquitination. Recently, the addition of a single ubiquitin to one (monoubiquitination) or multiple (multiubiquitination) protein sites has been reported. Also recently, BRCA1-BARD1 has been found to catalyze formation of Lys-6-linked polyubiquitin chains that differ from conventional ones, which are deubiquitinated in vitro by purified 26S proteasome instead of being targeted for degradation.

“Ubiquitination suppression” means complete or partial prevention of the ubiquitination.

As used herein, a “RNA polymerase” refers to an enzyme that synthesizes mRNA in 5′ to 3′ direction using a DNA strand as a template. The RNA polymerases include naturally occurring RNA polymerases and variant enzymes having the above-mentioned activity. Examples of such enzymes include RNA polymerase I, RNA polymerase II and RNA polymerase III.

As used herein, “common subunit” refers to a subunit shared by all RNA polymerases (e.g., RNA polymerase I, RNA polymerase II and RNA polymerase III).

As used herein, a “DNA-damaging environment” refers to an environment that causes conformational change in DNA molecules by radiation or the like, specifically, environments that inhibit any one of DNA synthesis, transcription of DNA into RNA or subsequent protein translation and that cause inactivation of RNA polymerase, inactivation of DNA polymerase, inactivation of DNA ligase, attenuation of nucleotide excision repair capacity, DNA methylation or the like. Examples of such DNA-damaging environments include ultraviolet (UV) irradiation, X-ray irradiation, chemical agents and active oxygen.

### 2. Ubiquitination of Common Subunit of RNA Polymerases by BRCA1-BARD1

Although BRCA1 biochemically functions as a ubiquitin ligase, its biological significance, especially in the DNA damage response, is little known.

We identified a mechanism underlying UV hypersensitivity in BRCA1 deficient cells. Failure of BRCA1 to ubiquitinate RPB8 resulted in UV sensitivity as well as prolonged phosphorylation of RNA polymerase II. These results not only emphasize the significance of BRCA1 's ubiquitin ligase activity in the DNA damage response, but they also enable further analysis of the roles that RNA polymerases may play in carcinogenesis. Further, our results could be applied clinically by providing a means to predict the sensitivity of breast cancers to DNA damaging anti-cancer agents, which can lead to therapeutic success of the cancers.

BRCA1 localizes to discrete nuclear foci during S phase. After DNA damage, BRCA1 is phosphorylated by ATM/ATR family kinases (Cortez et al., 1999; Tibbetts et al., 2000), and the BRCA1 foci disperse within 30 minutes (Scully et al., 1997b). These foci gradually reassemble into different foci where BRCA 1 cooperates with the Rad50-Mre11-Nbs1 complex (Zhong et al., 1999) or Rad51 and PCNA (Scully et al., 1997b) to repair the damaged DNA. The foci begin to appear approximately one hour after DNA damage has occurred, reach their peak after 6 to 8 hours, and remain until 12 hours post-damage (Zhong et al., 1999). For the most part, the S-phase foci are composed of BRCA1 and processive, hyperphosphorylated pol II, which dissociate upon DNA damage (Krum et al., 2003).

Pol II plays a critical role in the transcription-coupled DNA repair pathway. Pol II rapidly recognizes damaged DNA sites and signals damage by stalling at the sites. Pol II is replaced with CSA (Cockayne syndrome group A gene product) and CSB (Cockayne syndrome group B) followed by recruitment of TFIIH (Transcription factor IIH) and a complex including XPG (xeroderma pigmentosum group G gene product), XPF (xeroderma pigmentosum group F gene product), and ERCC1 (excision repair cross-complementing rodent repair deficiency, complementation group 1) that provide the nucleotide excision repair function (van den Boom et al., 2002). In intact cells, the hyperphosphorylated form of pol II is then dephosphorylated in order to be incorporated into the preinitiation complex at new regions.

Because BRCA1 associates with the processive pol II complex (Krum et al., 2003), BRCA1 should co-exist with the pol II complex when it is stalled in the cell. Therefore, BRCA1 appears to act as a sensor for the DNA damage (Lane, 2004). However, the early stage events that occur after DNA damage, but before BRCA1 dissociates from the S-phase foci (including the role of BRCA1 to the stalled pol II) was not clear.

The present invention demonstrates that BRCA1 polyubiquitinates a component of the pol II complex, RPB8, at this early stage after DNA damage. The timing of this event coincides with the period before BRCA1 dissociation from the stalled pol II. Importantly, a ubiquitin-resistant mutant of RPB8, which is capable of forming active RNA polymerase, remained bound to the hyperphosphorylated form of RPB1 for 6 hours after UV irradiation. This suggests that RPB8 ubiquitination by BRCA1 triggers dissociation of pol II from the damaged DNA.

It is well known that cells with impaired BRCA1 function display hypersensitivity to a range of DNA damaging agents including IR and UV irradiation (Abbott et al., 1999; Venkitaraman, 2002). However, the mechanism underlying this phenomenon is not fully understood. Although the failure of checkpoint function is a possible mechanism responsible for the hypersensitivity, it has been reported that neither selective abrogation of the S-phase checkpoint nor selective abrogation of the G2 checkpoint itself results in decreased cell survival after DNA damage (Xu et al., 2002a; Xu et al., 2002b). Therefore, it has been proposed that some function of BRCA1 other than S-phase or G2 cell cycle control may affect cell survival after DNA damage (Xu et al., 2002b).

The present invention demonstrated the UV sensitivity of the cells stably expressing a ubiquitin-resistant mutant of RPB8. Thus, the present invention provides a function of BRCA1 that may compensate for this conventional theoretical defect.

Prolonged hyperphosphorylation of RPB 1 was observed after UV irradiation of the pol II complex that contained the mutant RPB8. This is analogous to the form characterized as a stalled polymerase at damaged sites (Rockx et al, 2000; van den Boom et al., 2002), and is an extremely cytotoxic ramification of DNA damage (van den Boom et al., 2002). Further, transcriptional blockage triggered by UV irradiation is a potent inducer of apoptosis (Ljungman and Zhang, 1996).

It is interesting that there are considerable amount of the endogenous wild-type RPB8 expression in the ubiquitin-resistant RPB8 mutant cells (FIG. 7A). This indicates that only a partial interfere of RNA polymerase recovery by silencing a gene critical for cell survival is enough to induce cell death. Alternatively, pol II complexes containing mutant RPB8 stalled at the damaged site could subsequently cause additional stuck of following wild-type complexes. In support of this idea, induction of local damage by microbeam UV-irradiation in the nucleus led to transcription inhibition throughout the nucleus (Takeda et al., 1967).

Recently, ubiquitination of phosphorylated RPB 1 by BRCA1-BARD 1 has been reported by two groups (Kleiman et al., 2005; Starita et al., 2005). One group showed selective ubiquitination of the Ser5 hyperphosphorylated form of RPB 1-CTD (C-terminal domain of RPB1) (Starita et al., 2005). This ubiquitination is likely different from the previously reported ubiquitination of RPB 1 after DNA damage, since mutation of all 8 Lys residues within RPB1 's CTD did not affect its ubiquitination after UV (Ratner et al., 1998). The other group presented ubiquitination of phosphorylated RPB1 within the polymerase complex (Kleiman et al., 2005). Under their conditions, truncated, phosphorylated RPB 1-CTD fragment was not detectably ubiquitinated. Because double knockdown of BRCA1 and BARD1 restored the expression level of the phospholyrated pol II that had been repressed by UV irradiation, it was proposed that BRCA1-BARD1 could initiate the degradation of stalled RPB1. However, the BRCA1/BARD1 double knockdown did not detectably affect RPB 1 ubiquitination after UV irradiation.

Therefore, the restored expression level of the phospholyrated pol II by BRCA1/BARD1 double knockdown was possibly due to an indirect effect (Kleiman et al., 2005).

When coupled with our results the increased expression level of the phosphorylated RPB1 after UV irradiation was possibly due to failure of RPB8 ubiquitination (FIG. 8). Nonetheless, the in vitro ubiquitination of phosphorylated RPB1 by BRCA1-BARD1 (Kleiman et al., 2005) strongly supports its direct role.

Accordingly, RPB8 ubiquitination caused by BRCA1 appears to halt transcription upon DNA damage to prevent cell apoptosis.

DNA in BRCA1-deficient cell lines is sensitive to IR or UV irradiation while sensitivity to UV irradiation is also seen in ubiquitin-resistant RPB8 cell lines (i.e., mutants that do not effect ubiquitination). These results indicate that RPB8 ubiquitination via BRCA is involved in UV resistance in cells.

Further, RPB 1 hyperphosphorylation due to prolonged UV irradiation is detected in cells resistant to RPB8 ubiquitination

Experiment by Kleim et al. suggest that RPB1 phosphorylation of BRCA1-BARD 1-deficient cell line by UV irradiation is due to an indirect effect.

Thus, results obtained in the examples described below strongly suggest the relationship between RPB 1 phosphorylation and RPB8 ubiquitination via BRCA1-BARD1. In other words, RPB8 ubiquitination by BRCA1-BARD1 ubiquitinates and degrades phosphorylated RPB1 as well. Data obtained in these examples suggest that once DNA is damaged, for example, by UV irradiation, dissociation from polymerase II is caused, which halts the transcription and the like.

The key to solving this problem may be to analyze the timing of RPB 1 ubiquitination in vivo. Previously reported RPB 1 ubiquitination occurred two hours after UV irradiation, when BRCA1 should already be dissociated from pol II and relocalized to the DNA repair machineries (Cortez et al., 1999; Tibbetts et al., 2000).

It is possible that early after DNA damage, RPB1 and RPB8 could be transiently ubiquitinated by BRCA1 at the same time, and it may result in dissociation of the pol II holoenzyme from the damaged DNA site. In support of this, RPB 1 directly interacts with RPB8 in the pol II complex (Cramer et al., 2001). The RPB1 ubiquitination and degradation occurred in late phase could be mediated by other E3 ligases, such as Rsp5 (Huibregtse et al., 1997) (Beaudenon et al., 1999).

It is noteworthy to mention that there are two single nucleotide polymorphisms (SNPs) in the translated region of human RPB8 in NCBI database (http://www.ncbi.nlm.nih.gov/entrez/querv.fcgi?CMD=search&DB=snp), namely, a polymorphism having lysine at position 13 of the amino acid sequence of RPB8 substituted with aspartic acid (K13E) and a polymorphism having alanine at position 19 of the amino acid sequence of RPB8 substituted with glycine (A19G).

RPB8 ubiquitination by BRCA1 was slightly reduced when K13 is mutated (FIG. 6). A19G substitution may also affect the ubiquitination if it exists immediately before two consecutive lysine residues, K20/K21.

Because substitution of 5 Lys residues with Arg residues in the amino acid sequence of RPB8, despite its less expression compared to endogenous wild-type protein, causes hypersensitivity to DNA damage, it is possible that the RPB8 SNPs cause mild genetic instability resulting in cancer. Alternatively those SNPs may cause hypersensitivity to anti-cancer drugs. Clinical relevance of the ubiquitination capacity of these SNPs is one of the most important matters to be clarified.

It is noteworthy that RPB8 is shared by all three classes of RNA polymerases (Briand et al., 2001; Shpakovski et al., 1995). While pol II synthesizes mRNA, which is only about 5% of all RNAs, pol I and pol III synthesize rRNA, tRNA and all short untranslated RNAs, making up the remaining 95% of all RNAs. Therefore, modification of those complexes, rather than pol II, might enormously influence cellular conditions. For example, recent studies have revealed important roles for pol III transcription in cancer development (White, 2004).

Whereas RPB 1 has been intensively studied, the role of RPB8 in the DNA damage response has been poorly understood. According to the present invention, the ubiquitination of RPB8 mediated by BRCA1 in response to either DNA damaged or undamaged state provides additional evidence for the role of RNA polymerases in carcinogenesis as well as new insight into the tumor suppressor functions of BRCA 1.

Thus, the present invention can be applied to the field for controlling apoptosis and transcription by ubiquitination of RNA polymerase, where sensitivity of a cell to DNA damage can be changed by controlling RPB8 ubiquitination via BRCA1. This may be advantageous in treating cancer with drugs causative of DNA damage.

### 3. Pharmaceutical Composition Containing BRCA1-BARD

The present invention relates to a method and a pharmaceutical composition for treating cancer, comprising a gene encoding BRCA1-BARD1 or BRCA1-BARD. BRCA1-BARD1 ubiquitinates RPB8 to suppress cancer. Thus, the pharmaceutical composition of the invention can be used for treating cancer.

The pharmaceutical composition of the invention can be applied, for example, to cell proliferative diseases such as cancer. The pharmaceutical composition of the invention may be applied to either a single or complicated multiple disease case.

If the pharmaceutical composition of the invention is to be used for treating cancer, it can be applied to nonlimiting types of cancer including brain tumor, tongue cancer, pharynx cancer, lung cancer, breast cancer, esophagus cancer, stomach cancer, pancreas cancer, biliary tract cancer, gallbladder cancer, duodenal cancer, colorectal cancer, liver cancer, uterus cancer, ovarian cancer, prostate cancer, kidney cancer, bladder cancer, rhabdomyosarcoma, fibrosarcoma, osteosarcoma, chondrosarcoma, skin cancer, leukemias (e.g., acute myeloid leukemia, acute lymphatic leukemia, chronic myeloid leukemia, chronic lymphatic leukemia, adult T cell leukemia and malignant lymphoma).

The cancer may be any one of a primary lesion, a metastatic lesion and a concurrent lesion.

The pharmaceutical composition of the invention is used in a form such that BRCA1-BARD is intracellularly incorporated into the affected site or the tissue of interest.

The pharmaceutical composition of the invention may be administered in either oral or parenteral dosage form. Oral dosage forms for administration are suitably available in tablets, pills, sugar-coated forms, capsules, liquid forms, gel, syrups, slurry and suspension. Examples of parenteral dosage forms include transpulmonary forms (e.g., forms used with a nebulizer), transnasal forms, transdermal forms (e.g., ointments and creams) and injectable forms. Injectable forms may be systemically or locally administered in either a direct or an indirect way to the affected site, for example, through intravenous injection such as intravenous drip, intramuscular injection, intraperitoneal injection or subcutaneous injection.

In addition to a direct administration via injection, the pharmaceutical composition of the invention as a gene therapeutic drug may be used, for example, by administering a vector incorporating the nucleic acid. In this case, BRCA1-BARD1 may be used in a form where BRCA1 and BARD are co-expressed or as a fusion gene encoding a BRCA1-BARD1 complex.

Examples of such vectors include adenovirus vectors, adeno-associated virus vectors, herpesvirus vectors, vaccinia virus vectors, retrovirus vectors and lentivirus vectors. Use of these virus vectors allows efficient administration.

It is also possible to administer a phospholipid vesicle such as liposome carrying the pharmaceutical composition of the invention. Vesicles carrying the pharmaceutical composition of the invention are transfected into predetermined cells by lipofection. The resulting cells are systemically administered, for example, intravenously or intra-arterially. The cells may also be locally administered, for example, to brain. In order to introduce the pharmaceutical composition of the invention into a tissue or an organ of interest, commercially available gene transfer kits (e.g., AdenoExpress: Clontech) can be used. Although phospholipids, cholesterols or nitrogen-containing lipids can be used as the lipid for preparing liposome composition, phospholipids are generally favorable which include naturally-occurring phospholipids such as phosphatidylcholine, phosphatidylserine, phosphatidylglycerol, phosphatidylinositol, phosphatidylethanolamine, phosphatidic acid, cardiolipin, sphingomyelin, egg-yolk lecithin, soybean lecithin and lysolecithin, and hydrogenated forms thereof obtained by conventional techniques. Moreover, synthetic phospholipids such as dicetyl phosphate, distearoyl phosphatidylcholine, dipalmitoyl phosphatidylcholine, dipalmitoyl phosphatidylethanolamine, dipalmitoyl phosphatidylserine, eleostearoyl phosphatidylcholine and eleostearoyl phosphatidylethanolamine may be used.

Liposome can be prepared according to any conventional methods as long as the gene is retained, for example, the reverse phase evaporation technique (Szoka, F. et al., Biochem. Biophys. Acta, Vol. 601559 (1980),), the ether infusion technique (Deamer, D. W.: Ann. N.Y. Acad. Sci., Vol. 308250 (1978)) or the surfactant technique (Brunner, J. et al.: Biochim. Biophys. Acta, Vol. 455322 (1976)).

Lipids containing these phospholipids can be used alone or two or more of them can be used in combination. Use of those that intramolecularly include atoms with a cationic group, such as ethanolamine or choline can increase the binding rate of a negatively charged gene. In addition to a main phospholipid, known additives such as cholesterols, stearylamines and α-tocopherols can be used for the formation of liposome. To the resulting liposome, a membrane fusion promoter, for example, Sendai virus, inactivated Sendai virus, a membrane fusion promoting protein purified from Sendai virus or polyethylene glycol can be added for the purpose of promoting the intracellular uptake by the cells of the affected part or the tissue of interest.

The pharmaceutical composition of the invention can be formulated according to a typical method, which may include a pharmacologically acceptable carrier. Such a carrier may be an additive, examples being water, pharmacologically acceptable organic solvents, collagen, polyvinyl alcohol, polyvinylpyrrolidone, carboxy vinyl polymer, sodium carboxymethyl cellulose, sodium polyacrylate, sodium alginate, aqueous dextran, sodium carboxymethyl starch, pectin, methylcellulose, ethylcellulose, xanthan gum, gum arabic, casein, agar, polyethyleneglycol, diglycerine, glycerine, propylene glycol, petrolatum, paraffin, stearyl alcohol, stearic acid, human serum albumin, mannitol, sorbitol, lactose, and pharmacologically acceptable surfactants.

Said additives are selected alone or in any convenient combination according to the dosage form of the therapeutic drug of the invention. For example, for use as an injectable formulation, purified BRCA1-BARD or the gene thereof is dissolved in a solvent (e.g., saline, buffer or glucose solution), to which Tween 80, Tween 20, gelatin, human serum albumin or the like is added for use. Alternatively, it may be a lyophilized form that can be dissolved before use. Examples of diluents for lyophilization include sugars such as mannitol, glucose, lactose, sucrose, mannitol and sorbitol, starches from corn, wheat, rice, potato or other plants, celluloses such as methylcellulose, hydroxypropylmethyl cellulose and sodium carboxymethylcellulose, gums such as gum arabic and tragacanth gum, gelatin and collagen.

If desired, disintegrants or solubilizers such as cross-linked polyvinylpyrrolidone, agar, alginic acid or salt thereof (e.g., sodium alginate) can be used.

A given dose of the pharmaceutical composition of the invention differs depending on age, sex, condition, administration route, number of doses and dosage form. The administration method should be selected appropriately according to the age and the condition of the patient. An effective dose given is such that the symptom or condition of the patient is alleviated. The therapeutic effect of the pharmaceutical composition can be determined by a standard pharmaceutical procedure, for example, ED50 (dose that is therapeutically effective for 50% of the population) or LD50 (dose that is lethal for 50% of the population) in cell cultures or experimental animals.

The dose ratio between therapeutic and toxic effects is the therapeutic index that may be expressed in ED50/LD50. The given dose of the pharmaceutical composition of the invention is, for example, 0.1 μg-100 mg/kg, preferably 1-10 μg/kg at a time. The above-mentioned therapeutic drug, however, should not be limited to these doses. The given dose upon administering adenovirus is approximately 10⁶-10¹³ once a day for 1-8 weeks of administration. The pharmaceutical composition of the invention, however, should not be limited to these doses.

Hereinafter, the present invention will be described more specifically by way of examples. The present invention, however, should not be limited to these examples.

### EXAMPLES

### 1. Materials and Methods

Firstly, different types of experimental procedures employed in the examples will be described below.

(1) 2D-DIGE

2D-DIGE analysis was carried out according to the manufacturer's instructions (Amersham). Briefly, cells either untreated or treated with epirubicin were harvested, rinsed three times in cold wash buffer (10 mM Tris-HCl, pH 8.0 and 5 mM MgAc), and solubilized by sonication in lysis buffer (7 M urea, 2 M thiourea, 4% CHAPS and 30 mM Tris-HCl, pH 8.0). The lysates were centrifuged at 12,000 g for 10 min at 4° C., and the supernatants were collected. Fifty micrograms each of protein from any of untreated, epirubicin-treated, or a 1:1 mixture of both samples were labeled with Cy3, Cy5 or Cy2 fluorescence dye, respectively.

All three differently labeled samples were mixed and applied on 24-cm sigmoidal immobilized pH gradient (IPG) isoelectric focusing gel strip (pH range 3-10; Amersham) for separation by first dimension electrophoresis. Isoelectric focusing was carried out with MultiPhor II (Amersham).

The IPG strips were then equilibrated with a solution containing 6 M urea, 30% glycerol, 2% SDS, 0.05 M Tris-HCl, pH 8.0 and 0.5% DTT for 15 minutes and blocked by substituting the DTT with 4.5% iodoacetamide in the equilibrating buffer.

The focused proteins were then separated by 12.5% polyacrylamide gel (SDS-PAGE). The 2D separation gels were scanned with Typhoon 9400® image analyzer (Amersham). The protein spots that were significantly different between the control sample and the epirubicin-treated sample were identified by Decyder® version 5.01 software (Amersham) using the Cy2-labeled mixed proteins as internal controls.

For protein identification, a 1 mg mixture of untreated and epirubicin-treated cells was resolved by a 2D gel as described above and stained with Sypro Ruby (Molecular Probe). The protein spots of interest were excised from the gel and digested with trypsin using the In Gel Digest Kit (Millipore) according to the manufacturer's instructions. The peptide fragments were subjected to LC/MS/MS analysis as described (Nishikawa et al., 2004). The Mascot software program (Matrix Science, London, UK) analyzed the collision-induced dissociation spectra acquired by searching the National Center for Biotechnology Information (NCBI) protein databases.

(2) Plasmid

Full length cDNA for human RPB8 was amplified by PCR from a HeLa cell cDNA library using Pfx polymerase (Stratagene). It was subcloned into pcDNA3 or pGEX vectors in-frame with the N-terminal FLAG or GST tag, respectively. Known mammalian expression plasmids for BRCA 1, BARD 1, ubiquitin and their mutants were used (Hashizume et al., 2001; Nishikawa et al., 2004).

The point mutation was employed to substitute Lys residue of RPB8 with Arg residue by site-directed mutagenesis (Stratagene). All plasmids used were verified by DNA sequencing.

(3) Cell Cultures and Transfections

Breast cancer T47D, MCF7, HCC1937 cell lines, cervical cancer HeLa cell line, and transformed human kidney 293T cell line were cultured in Dulbecco's Modified Eagle's Medium (DMEM) supplemented with 10% fetal calf serum and 1% antibiotic-antimycotic agent (Life Technologies, Inc) in 5% CO₂ at 37° C. Normal human epithelial MCF 10A cells were grown in DMEM/Ham's F12 (1:1) medium supplemented with 2.5% fetal calf serum, 100 ng/ml cholera toxin, 20 ng/ml EGF, 500 ng/ml hydrocortisone, 10 μg/ml insulin and 1% antibiotic-antimycotic agent. For epirubicin treatment, cells were incubated for the indicated time in medium containing 0.2 μg/ml epirubicin (Pfizer). To examine the half-life of proteins in vivo, cells were incubated with 10 μg/ml of cyclohexamide (Wako) for the indicated time. 293T cells were transfected using the standard calcium phosphate precipitation method. For each transfection, total plasmid DNA was adjusted to be equal by adding pcDNA3 empty vector. To generate cell lines that stably expressed either wild-type or mutant FLAG-RPB8, pcDNA3 encoding each protein was transfected into HeLa cells using FuGENE6® (Roche).

Forty-eight hours after transfection, cell suspensions were diluted, seeded, and selected with 0.5 mg/ml G418. Colonies of the transformants were obtained after two weeks of culture, and the cloned cells were further amplified and maintained in 0.25 mg/ml G418. For UV irradiation studies, cells were washed with PBS, irradiated with UV light (254 nm; UVP Inc, Upland, Calif.) at the indicated doses, and grown in fresh medium for indicated time periods. Cell viabilities were analyzed either by phase contrast microscopy, trypan blue exclusion measurements, or Lillie's crystal violet staining.

(4) Antibodies

Mouse monoclonal antibodies to HA (12CA5, Boehringer, Mannheim), Myc (9E10, BabCo), FLAG (M2, Sigma), ubiquitin (FK1, Affiniti), phospho-S5 RPB1 (H14, COVANCE), α- and β-tubulin (DMIA+BMIB, Neomarkers), and actin (C2, Santa Cruz) as well as rabbit polyclonal antibodies to BRCA1 (C20, Santa Cruz) and RPB1 (8WG16, COVANCE) were purchased commercially. Rabbit polyclonal antibodies to BARD1 and RPC155 were generous gifts from Dr. Richard Baer (Colombia University) and Dr. Nouria Hernandez (Cold Spring Harbor Laboratory), respectively. Anti-RPB8 rabbit polyclonal antibody was generated against full-length human GST-RPB8 as antigen. Crude serum from the immunized rabbit was incubated with GST-conjugated glutathione agarose beads to purify away the anti-GST antibody followed by protein A agarose chromatography. The pre-immunized rabbit serum as control was treated similarly.

(5) siRNA

SMART Pool® BRCA1 siRNA mix and control siRNA mix were purchased from Dharmacon Research, Inc. RNA duplexes (final concentration 50 nM) were transfected into the cells with Oligofectamine® (Invitrogen) according to the manufacturer's instructions. Retrovirus expressing shRNA that targets BRCA1 mRNA sequence 5′CUAGAAAUCUGUUGCUAUG3′ (SEQ ID NO:1) was created by co-transfecting 293T cells with pGP vector, pE-ampho vector and pSINsi-hU6 retrovirus vector that has previously been subcloned with oligonucleotide 5′ GATCCGCTAGAAATCTGTTGCTATGTTCAAGAGACATAGCAACAGATTTCTA GCTTTTTTAT3′ (SEQ ID NO:2) according to manufacturer's protocol (TaKaRa). Oligonucleotide 5′ GATCCGTAAGGCTATGAAGAGATACTTCAAGAGAGTATCTCTTCATAGCCT TACTTTTTTAT3′ (SEQ ID NO:3) was used for the retrovirus expressing control shRNA.

The supernatant containing the retrovirus was stored at −80° C. until use. For infection, HeLa cells cultured in 150-mm plates were cultured with 15 ml of a 1:10 mixture of virus supernatants and fresh culture medium containing 8 μg/ml Polybrene (Sigma). Cells were analyzed 48 hours after transfection or infection.

(6) Immunoprecipitation and Immunoblotting

Immunoprecipitation and immunoblotting methods, including the detection of in vivo ubiquitinated substrates using boiled 1% SDS-containing buffer, were carried out according to a known method (Nishikawa et al., 2004; Sato et al., 2004).

For the immunoblotting analysis following 2D gel electrophoresis, cells were lysed with 7 M urea/2 M thiourea-containing buffer as described above.

(7) GST Pull-Down Assays

GST or GST-fused full length RPB8 protein was purified by glutathione affinity chromatography following expression in BL21 bacteria using standard methods. Four micrograms of the GST fusion protein was mixed with 4 μg of His-BARD1¹⁴⁻¹⁸⁹ and 25 μl of glutathione-agarose beads in 1 ml of a buffer containing 50 mM Tris-HCl (pH 7.5), 0.5% NP-40, 150 mM NaCl, 50 mM NaF and 1 mM dithiothreitol. After rotation at 4° C. for 2 hours, GST fusion proteins bound to glutathione-agarose beads were washed three times and subsequently eluted with SDS-PAGE loading buffer.

(8) Run-Off Transcription Assay

The run-off transcription assay followed a known method (Krum et al., 2003). Briefly, the run-off template was created by annealing 50 pmol each of a 65-mer oligonucleotide 5 ′ATTGGGTAAAGGAGAGTATTTGAGCGGAGGACAGTACTCCGGGTCCCCCCC CCCCCCCCCCCCCC3′ (SEQ ID NO:4) and a complementary 45-mer oligonucleotide 5′GACCCGGAGTACTGTCCTCCGCTCTTTTACTCTCCTTTACCCAAT3′ (SEQ ID NO:5) in a 200 μl annealing mixture (20 mM Tris (pH 7.4), 1 mM EDTA and 0.2 M NaCl).

Run-off transcription reactions (20 μl) contained 8.25 mM MgCl₂, 5 μg of bovine serum albumin, 250 nM NTPs, 5 units of RNase inhibitor, 50 ng of poly (dI-dC), 0.05% Nonidet P-40, 1 pmol of annealed oligonucleotides, and 0.5 μCi of [α-32P] CTP.

Equilibrated FLAG-RPB8 immunocomplexes bound to M2 beads (10 μl) were added to the reactions (20 μl) and incubated for 40 min at 30° C. and stopped with 50 μl of PK buffer (300 mM sodium acetate, 0.2% SDS, 10 mM EDTA, 100 ng of tRNA, and 10 μg of proteinase K). Reactions were then incubated at 55° C. for 20 min, transcripts were extracted with phenol/chloroform, and precipitated with ethanol. Single-stranded RNA transcripts were resolved under denaturing conditions on 12% polyacrylamide/urea gels and scanned with Typhoon 9400® (image analyzer (Amersham).

### 2. Results

(1) Identification of RPB8 as a Protein Modified in BRCA1 Positive Cells Following Epirubicin Treatment

To search for candidate substrates for the BRCA1-BARD1 E3 ligase reactive to DNA damage, we employed fluorescence two-dimensional difference gel electrophoresis (2D-DIGE) technology.

Breast cancer-derived, BRCA1-positive T47D cells and BRCA1-deficient HCC1937 cells were incubated for 3 hours in the presence of epirubicin, a topoisomerase II inhibitor (that induces DNA double strand breaks). Cells were lysed with 7M urea/2M thiourea-containing buffer, and the proteomes were compared with untreated cells as analyzed by 2D-DIGE method.

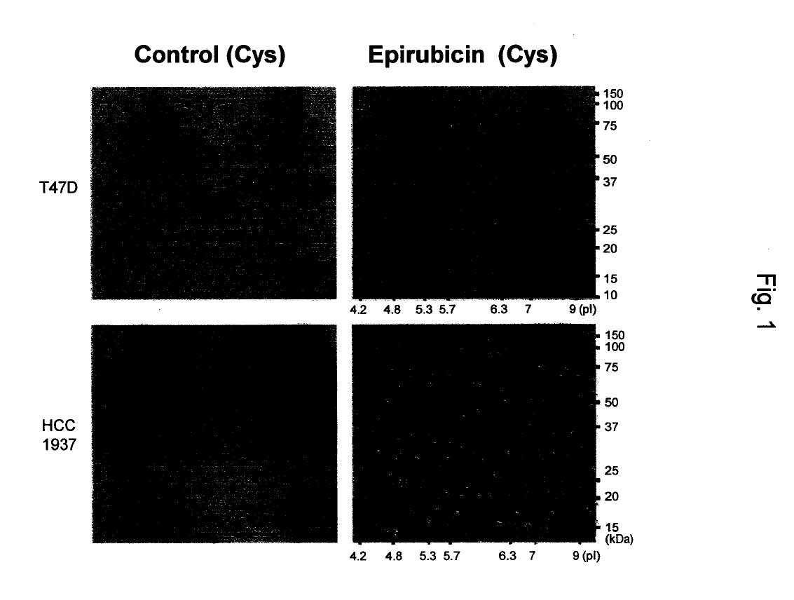

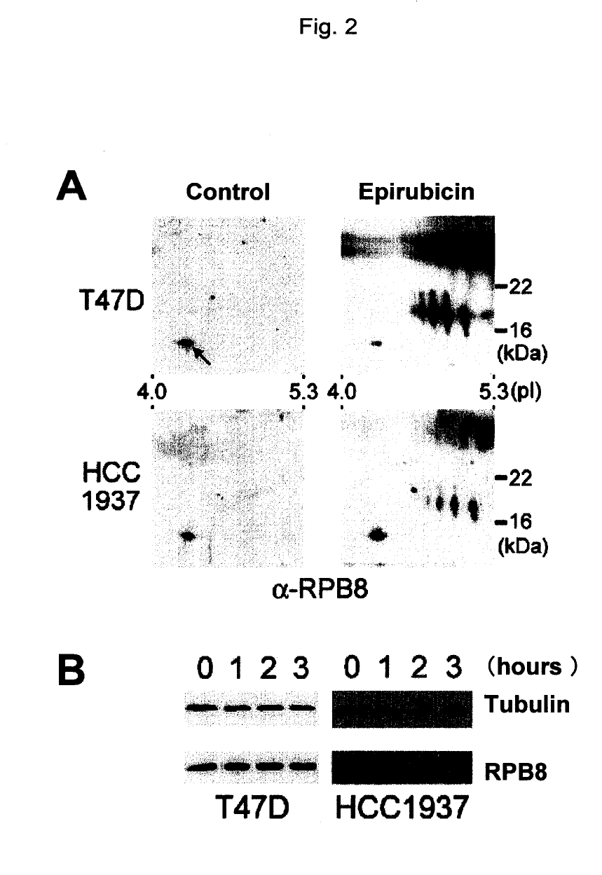

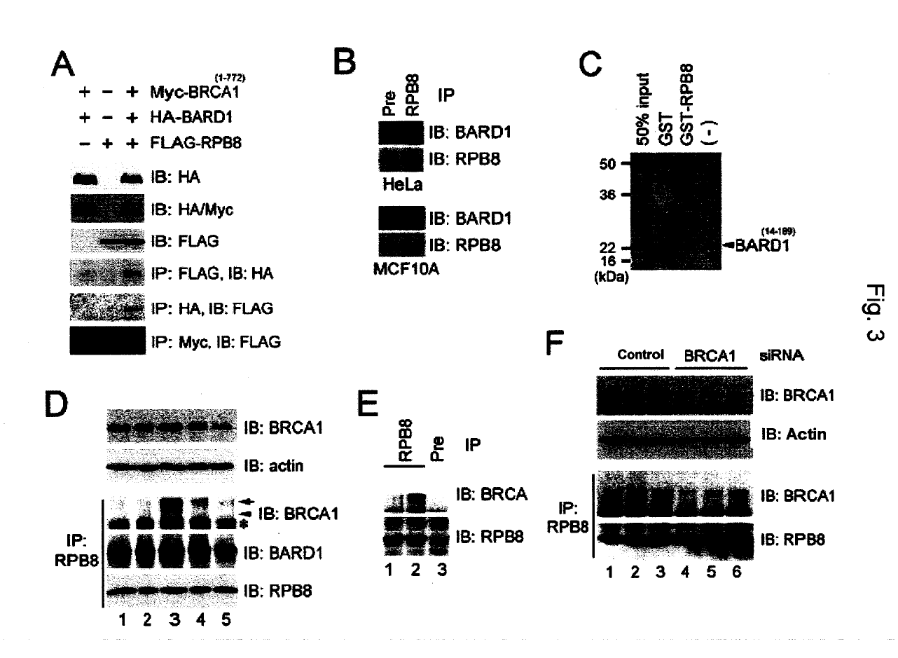

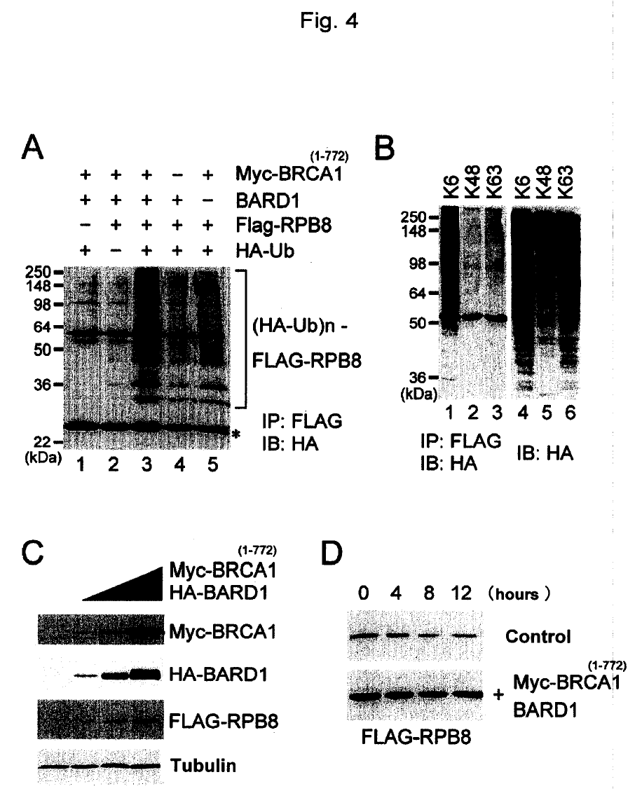

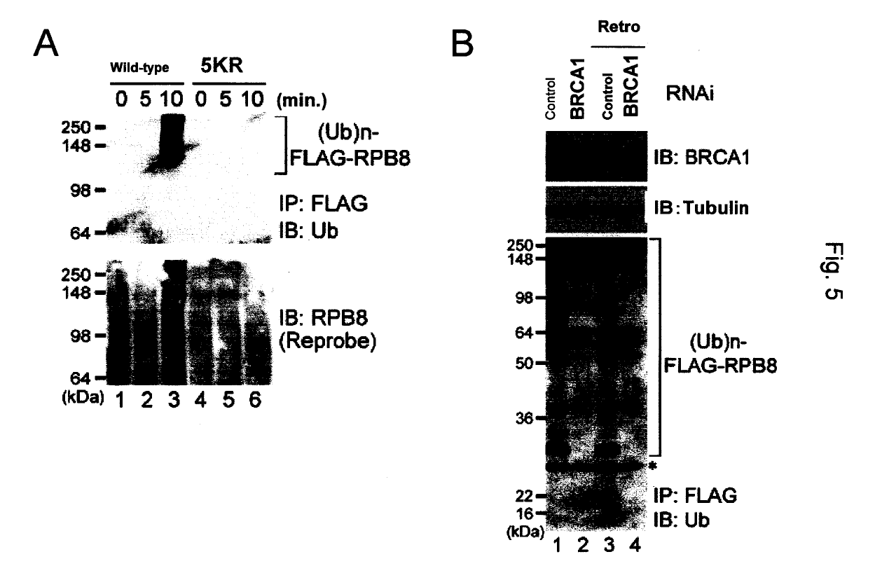

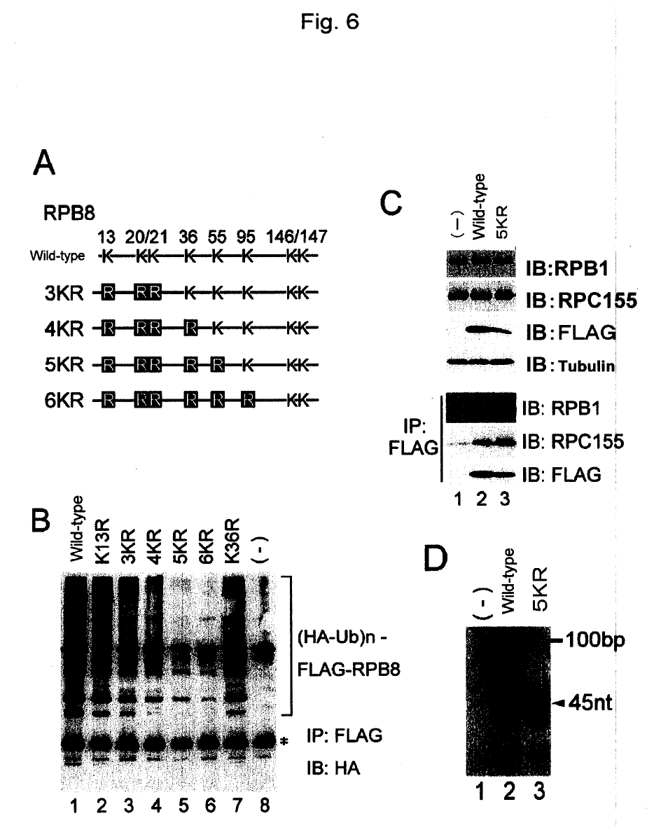

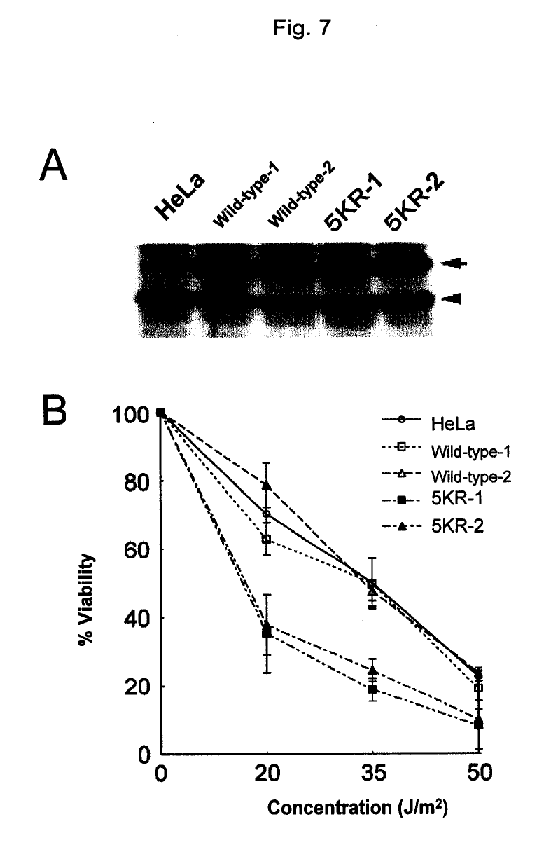

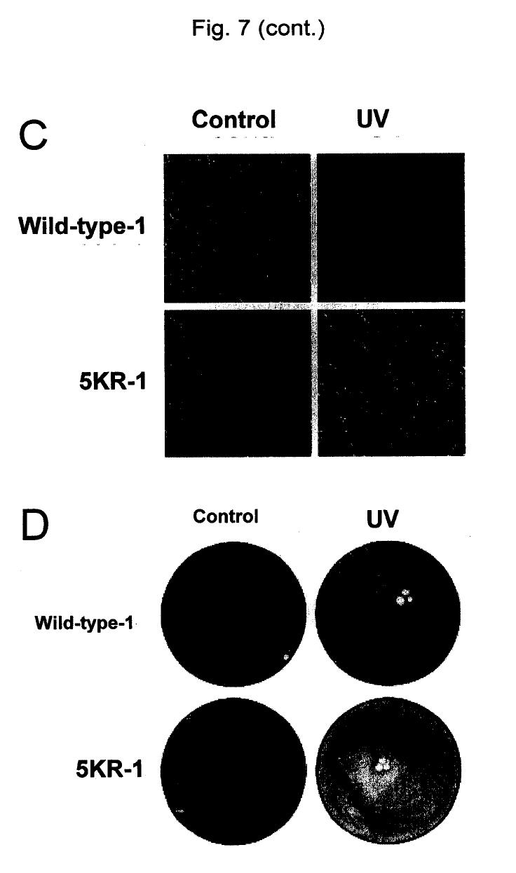

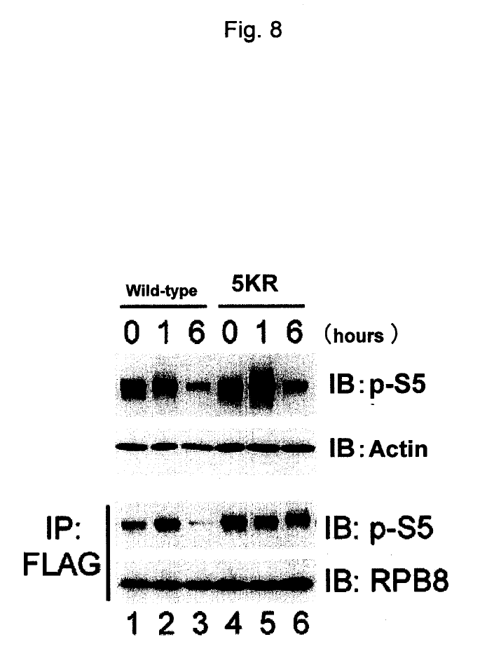

Interestingly, whereas the expression levels of only a few proteins were affected by the epirubicin treatment in T47D cells, the expression levels of approximately 100 types of proteins were altered in HCC1937 cells (FIG. 1, yellow arrows). Even more interestingly, two types of proteins whose expression levels were dramatically reduced in T47D cells showed no change in HCC1937 cells (FIG. 1, two red arrows at the lower-left corner). Similar results were detected in other BRCA1 intact cell lines. Therefore, we speculated that the reduction in the expression level could depend on the presence of BRCA1.

The protein spots were in-gel-digested and subjected to nanoscale capillary liquid chromatography-tandem mass spectrometry (LC/MS/MS) analysis. LC/MS/MS analysis revealed that the samples were RPB8, a common subunit of three types of RNA polymerases, and myosin light chain. RPB8 is a very acidic, small protein with a calculated molecular mass of 17.1 kDa and an isoelectric point of pI 4.34 (Shpakovski et al, 1995). Because the pol II holoenzyme interacts with BRCA1 and because RPB1, the largest subunit of pol II, is a potential substrate for the BRCA1-BARD1 E3 ligase, we focused on RPB8 for further analyses.

To confirm the mass spectrometry data, we generated an anti-GST-RPB8 rabbit polyclonal antibody for immunoblot analysis. Cells were treated with epirubicin and immunoblot analysis of the proteins resolved by 2D-gels verified that the protein spot was indeed RPB8. RPB8 was again severely reduced by epirubicin treatment only in T47D cells (FIG. 2A). Interestingly, several protein ladders reactive to anti-RPB8 antibody which migrated at positions of higher molecular weight and more basic pH appeared after epirubicin treatment. Since the ladders were not detectable with ruby stain, we concluded that these protein species existed in low amounts.

One possibility of lower amounts of these proteins was that they represented forms of RPB8 that were covalently modified with ubiquitin. However, when cells were lysed with 0.5% NP-40-containing buffer and resolved by one-dimensional SDS-PAGE, neither the reduction in RPB8 expression levels nor the protein ladder was detected by immunoblotting (FIG. 2B). This suggested that the reduced expression level of RPB8 observed by 2D gel analysis was not due to protein degradation, but rather to conversion of RPB8 into BRCA1-dependent covalently modified forms in response to DNA damage.

(2) Interaction of RPB8 with BRCA1-BARD1

In order to dissect the molecular basis of the observed RPB8 modification, we tested the interaction of RPB8 with BRCA1-BARD1 using exogenously expressed proteins.

293T cells were co-transfected with Myc-BRCA1¹⁻⁷⁷², HA-BARD1 and FLAG-RPB8, and BRCA1/BARD1 immunoprecipitates were probed for the presence of FLAG-RPB8. As a result, a significant amount of FLAG-RPB8 was detected in both anti-Myc and anti-HA immunocomplexes as compared to controls. Reciprocally, HA-BARD1 co-purified with FLAG-RPB8 immunocomplexes as well as controls were undetected (FIG. 3A).

Next, we tested if endogenous RPB8 could interact with BRCA1 and BARD1. A significant amount of BARD1 co-immunoprecipitated with RPB8 isolated from untreated HeLa cells as compared to controls (FIG. 3B, upper panel). The same results as that of MCF10A shown in the lower panel in FIG. 3B were observed with all cell lines tested including MCF7, T47D, and 293T.

Both BRCA1 and BARD1 interact with the pol II holoenzyme (Chiba and Parvin, 2002). Therefore it is possible that the holoenzyme bridges the interaction between BRCA1/BARD1 and RPB8. To examine whether the interaction between BARD1 and RPB8 is direct, we purified bacterially expressed recombinant GST-RPB8 and recombinant His-BARD1 (14-189) proteins. His-BARD1 (14-189) protein lacks a region of 13 amino acids at the N-terminus of full-length BARD1 amino acid sequence. GST-RPB8 was co-precipitated with His-BARD1 (14-189) but GST was undetected (FIG. 3C). This result suggests that RPB8 directly interacts with the N-terminus of BARD1.

BARD1 binds to BRCA1 to form an active E3 ligase. Although we detected a significant amount of BARD1 in RPB8 immunoprecipitates, we failed to detect BRCA1. We therefore speculated that BRCA1 might interact with BARD1 and RPB8 only under specific conditions such as DNA damage. To test this, we irradiated MCF10A cells with UV and harvested them at several time points after irradiation. The harvested cells were lysed, and the cell lysates were subjected to immunoprecipitation with anti-RPB8 antibody followed by immunoblotting with anti-BRCA1, anti-BARD1 or anti-RPB8 antibodies.

While BARD1 was easily detected in anti-RPB8 immunocomplexes at all time points analyzed, BRCA1 was specifically detected at 10 minutes and only faintly at 60 minutes after irradiation (FIG. 3D). FIGS. 3E and 3F show the results with MCF10A cells while the same results were observed with T47D, MCF7, HeLa and MDA-MB435 cells.

Curiously, the BRCA1 that co-purified with RPB8 migrated more slowly than BRCA1 from whole cell extracts. Therefore, we sought to verify that this protein was indeed BRCA1 through the use of siRNA.

T47D cells were transfected with either control siRNA or BRCA1 siRNA and then irradiated with UV 48 hours post-transfection.

The specific reduction in BRCA1 expression as compared to whole cell extracts was confirmed by immunoblotting (FIG. 3F, uppermost panel). Further, the BRCA1 protein detectable with RPB8 10 minutes after UV irradiation was eliminated with BRCA1 siRNA, but not with control siRNA (second bottom panel, compare Lanes 2 and 5). This confirms that the slowly migrating product in RPB8 immunoprecipitates is a modified form of BRCA1.

(3) RPB8 Ubiquitination and Stabilization by BRCA1-BARD1 In Vivo

We next tested whether RPB8 is ubiquitinated by BRCA1-BARD1 in vivo.

FLAG-RPB8 was co-expressed in 293T cells with HA-ubiquitin, Myc-BRCA1 (1-772) and BARD1. Cells were collected thirty-six hours after transfection and boiled in 1% SDS containing buffer, and FLAG-RPB8 was immunoprecipitated. Immunoblotting of the RPB8 precipitates resolved by SDS-PAGE using anti-HA antibody demonstrated a ladder characteristic of polyubiquitinated RPB8 (FIG. 4A).

Omission of FLAG-RPB8, HA-ubiquitin, Myc-BRCA1 (1-772) or BARD1 all abolished the RPB8 ladders supporting the idea of BRCA1-BARD1-dependent RPB8 ubiquitination.

BRCA1/BARD1 is the only known E3 ligase to catalyze Lys6-linked polyubiquitin chains (Morris and Solomon, 2004; Nishikawa et al., 2004; Wu-Baer et al., 2003). To demonstrate that the in vivo RPB8 ubiquitin ladders were directly due to BRCA1/BARD1 ligase activity, we verified that RPB8 was modified by ubiquitin through Lys6 linkages.

HA-tagged ubiquitins that have a single Lys residue available for conjugation were used for in vivo ubiquitination assays. As expected, BRCA1-BARD1-dependent RPB8 polyubiquitination was predominantly detected when HA-ubiquitin with only Lys6 available for conjugation, but not Lys48 or Lys63, was co-expressed (FIG. 4B). Hence, the results suggest that the in vivo RPB8 polyubiquitination observed is directly catalyzed by BRCA1-BARD1.

Lys6-linked polyubiquitination of the substrates, BRCA1 itself or NPM, due to BRCA1/BARD1 catalysis is not a signal for proteasomal degradation (Hashizume et al., 2001; Nishikawa et al., 2004; Sato et al., 2004). Therefore, we analyzed whether this was also the case with BRCA1/BARD1-dependent RPB8 ubiquitination. That is to say, we analyzed whether RPB8 ubiquitination as well was not a signal for proteasomal degradation.

The steady state level and the protein half-life of FLAG-RPB8, when co-expressed in 293T cells with Myc-BRCA1 (1-772) and BARD1, were examined. The steady state level of FLAG-RPB8 increased upon co-expression of BRCA1-BARD1 in a dose-dependent manner (FIG. 4C). Treatment of cells with cyclohexamide also demonstrates that BRCA1-BARD1 stabilizes RPB8 (FIG. 4D). These findings suggest that, like other substrates we have reported (Hashizume et al., 2001; Nishikawa et al., 2004; Sato et al., 2004), BRCA1-BARD1-mediated RPB8 ubiquitination affects the function of RPB8 through a non-proteolytic mechanism.

(4) BRCA1-Dependent RPB8 Ubiquitination Following UV Irradiation

BRCA1-mediated RPB8 ubiquitination prompted us to investigate the biological implications of this activity.

BRCA1 has long played a role in transcription-coupled DNA repair (Abbott et al., 1999; Le Page et al., 2000), and ubiquitination of the largest subunit of pol II, RPB1, in response to DNA damage has been shown (Beaudenon et al., 1999; Bregman et al., 1996; Kleiman et al., 2005; Lee et al., 2002; Starita et al., 2005). Therefore, we examined if RPB8 is ubiquitinated in response to DNA damage.

Rather than exposing cells continuously to epirubicin, we employed UV irradiation to accurately determine the timing of RPB8 ubiquitination after DNA damage. We established HeLa cell lines that stably express FLAG-RPB8 (FIG. 7A) and analyzed ubiquitination of anti-FLAG immunoprecipitates. Cells were collected at several time points after UV irradiation and boiled in 1% SDS containing buffers, and FLAG-RPB8 was immunoprecipitated. FLAG-RPB8 was then eluted from the antibody with FLAG peptide, and eluates were immunoblotted with anti-ubiquitin antibody. Because it has been reported that RPB1 ubiquitination occurs one to two hours after UV irradiation (Bregman et al., 1996; Kleiman et al., 2005; Ratner et al., 1998; Starita et al., 2005), we analyzed these time points. However, we did not detect any ubiquitination of FLAG-RPB8.

Instead, ubiquitinated FLAG-RPB8 readily appeared approximately 10 minutes after UV irradiation (FIG. 5A, upper panel, Lane 3). This timing is consistent with the conditions required for interaction of RPB8 with BRCA1, namely, response at 10 minutes after UV irradiation (FIG. 3D, Lane 3).

Reprobing the membrane with anti-RPB8 antibody verified that the detected ladder was ubiquitinated RPB8 (FIG. 5A, lower panel). No such ladder was detected when a HeLa cell line that stably expressed a ubiquitin-resistant mutant, FLAG-RPB8 5KR (described below) was used as a control (FIG. 5A, Lanes 4-6).

To verify that UV irradiation-induced RPB8 ubiquitination requires BRCA1, RNAi was again employed to knock down endogenous BRCA1 expression. Two techniques were employed for RNAi expression. In one technique, HeLa cells stably expressing FLAG-RPB8 were transfected with BRCA1-specific siRNA. In the other technique, we constructed a retrovirus engineered to express shRNA for BRCA1. Forty-eight hours after transfection or infection, cells were irradiated with UV (35J/m²) and then harvested 10 minutes later.

Both the siRNA-transfected and the shRNA-retrovirus-infected cells were successfully silenced for BRCA1 expression (>90% and >75% reduction, respectively) compared with their controls (FIG. 5B, upper panel). As expected, RPB8 ubiquitination after UV irradiation was dramatically reduced by BRCA1 knockdown in both cases (FIG. 5B, lower panel). Prominent doublet bands that migrated around 28-30 kDa completely disappeared upon BRCA1 knockdown. These results further support the idea that RPB8 is polyubiquitinated by BRCA1-BARD1 in an early phase after DNA damage.

(5) Ubiquitin-Resistant Form of RPB8 and its Relationship with Polymerase Activity

For the purpose of studying the physiological consequences induced by the BRCA1-mediated RPB8 ubiquitination following UV irradiation, we generated a mutant of RPB8 that is incapable of being ubiquitinated by BRCA1-BARD1. RPB8 possesses eight Lys residues in the whole protein. We first mutated a single Lys residue of RPB8 and tested its capacity to be ubiquitinated. However, RPB8 ubiquitination was not dramatically reduced by each of the single mutation (FIG. 6B, Lanes 2 and 7). Instead, the ubiquitination of RPB8 was reduced as the number of Lys to Arg substitutions increased. This result recapitulates what we observed during studies of BRCA1 autoubiquitination and of BRCA 1-mediated NPM ubiquitination.

When five of the eight Lys residues were substituted with Arg (5KR), RPB8 ubiquitination became undetectable (FIG. 6B, Lane 5), whereas the binding capacity of RPB8 to BRCA1-BARD1 was not reduced.

To confirm that the many mutations required to make RPB8 resistant to ubiquitination did not impair its fundamental function as a subunit of RNA polymerases, we verified that the 5KR mutant is capable of binding to RPB1 or RPC155 (the largest subunit of pol II and III, respectively) in vivo.

Wild-type FLAG-RPB8 or 5KR was transfected into 293T cells, and anti-FLAG immunocomplexes were isolated. Bound proteins were resolved by SDS-PAGE and analyzed by immunoblotting using anti-RPB1 or anti-RPC155 antibodies. A significant amount of both RPB1 and RPC155 were detected in the FLAG-5KR, as well as the wild-type immunocomplex (FIG. 6C).

We measured catalytic activity of anti-FLAG immunoprecipitates using a run-off transcription assay. The 5KR mutant immunocomplexes contained the ability to generate in vitro transcripts equal to that of wild-type immunocomplexes (FIG. 6D). Thus, the 5KR mutant of RPB8 constitutes a viable RNA polymerase complex in vivo that sustains its polymerase activity. This indicates that RPB8 ubiquitination by BRCA1-BARD1 does not require RNA polymerase activity.

(6) Sensitivity of Ubiquitin-Resistant Mutant of RPB8 to Uv

Stable expression of 5KR mutant of RPB8 does not result in RPB8 ubiquitination even in the presence of endogenous BRCA1.

On the other hand, BRCA1 deficiency causes sensitivity to DNA damage (Abbott et al., 1999; Ruffner et al., 2001; Shen et al., 1998). Since BRCA1-deficient cell lines do not result in RPB8 ubiquitination, it is possible that this could cause the same phenotype as 5KR mutants. Because RPB8 is ubiquitinated by BRCA1 after UV irradiation (FIG. 5), it is possible that failure of ubiquitination could cause the same phenotype. To test this possibility, we established HeLa cell lines that stably express the 5KR mutant of FLAG-RPB8.

Two clones each of the wild-type (WT-1 and WT-2) and of the 5KR mutant (5KR-1 and 5KR-2) cell lines were obtained. Expression of FLAG-RPB8 in these clones was confirmed (FIG. 7A). Using these cells, we examined if the expression of the mutant RPB8 affected cell survival after UV irradiation. Cell viability was determined by trypan blue exclusion 48 hours after irradiation.

The cell viabilities of the 5KR clones after 20 or 35 J/m² of UV irradiation were approximately 38% and 23% of untreated cells at 0 hours, respectively, whereas wild-type clones were approximately 72% and 53%, respectively (FIG. 7B). Parental HeLa cells exhibited viabilities similar to that of wild-type clones (FIG. 7B). Cells observed by phase contrast microscopy 48 hours after UV irradiation (35 J/m²) and culture plates stained with Lillie's crystal violet stain are shown in FIGS. 7C and 7D, respectively.

Thus, expression of a ubiquitin-resistant RPB8 appears to cause UV sensitivity of the cells.

(7) Ubiquitin-resistant mutant of RPB8 causes prolonged RPB1 phosphorylation after UV irradiation

We next addressed the mechanism underlying the UV sensitivity of the RPB8 5KR mutant cell line. In the transcription-coupled repair pathway for DNA damage, Cockayne syndrome A (CSA) and CSB proteins play a critical role to displace the RNA polymerase complexes from the damaged site. This allows the subsequent recruitment of the repair complex including TFIIH (Rockx et al., 2000; van den Boom et al., 2002). RPB1 becomes hyperphosphorylated on its C-terminal domain (CTD) during RNA elongation, and BRCA1 specifically interacts with this form, possibly as a sensor for DNA damage. When pol II remains at the damaged site, it retains its hyperphosphorylated status for approximately one hour after UV irradiation. As the complex is displaced by CSA/CSB, it is dephosphorylated (within 6 hours after UV irradiation) (Rockx et al., 2000). This process is important for cell survival, and prolonged stalling of pol II in its phosphorylated form at the damaged site is extremely cytotoxic (Ljungman and Zhang, 1996; van den Boom et al., 2002).

The UV sensitivity of the 5KR-mutant-expressing RPB8 cells prompted us to investigate if the 5KR mutant of RPB8 causes prolonged phosphorylation of RPB1 after UV damage.

To verify this point, HeLa cell lines stably expressing either wild-type or 5KR mutant FLAG-RPB8 were irradiated with UV. Cells were harvested 1 or 6 hours after UV irradiation and subjected to immunoblotting with H14 antibody that recognizes RPB1 phosphorylated at Ser5 of its CTD repeats.

In both wild-type and mutant cell lines, the amount of phosphorylated RPB1 slightly increased one hour after UV irradiation, and subsequently decreased at 6 hours after irradiation (FIG. 8, top panel).

The electrophoretic mobility of phosphorylated RPB1 was reduced after UV irradiation, indicating that the number of phosphorylation sites in each molecule was also increased. These results were consistent with stalled pol II and its subsequent displacement and dephosphorylation after UV irradiation as previously reported (Rockx et al., 2000). However, the phosphorylation status of RPB1 within FLAG-RPB8 5KR mutant immunoprecipitates remained phosphorylated at 6 hours after UV irradiation in contrast to the normal dephosphorylation event taking place in wild-type complexes as controls (FIG. 8, third panel). This suggests that the majority of RPB1 in 5KR cells interacted (to be exact, dephosphorylated) with the endogenous wild-type RPB8, while the RPB1 in the 5KR mutant complexes was not.

Together, the results are consistent with the hypothesis that RPB8 ubiquitination by BRCA1 has a critical role in displacing pol II from the damaged DNA site. This subsequently allows the polymerase to be dephosphorylated and recycled for incorporation into the next preinitiation complex. Without this process, stalled pol II causes cell death.

### REFERENCES

1) Abbott, D. W., Thompson, M. E., Robinson-Benion, C., Tomlinson, G., Jensen, R. A., and Holt, J. T. (1999). BRCA1 expression restores radiation resistance in BRCA1-defective cancer cells through enhancement of transcription-coupled DNA repair. J Biol Chem 274, 18808-18812.
2) Al-Wahiby, S., and Slijepcevic, P. (2005). Chromosomal aberrations involving telomeres in BRCA1 deficient human and mouse cell lines. Cytogenet Genome Res 109, 491-496.
3) Baer, R., and Ludwig, T. (2002). The BRCA1/BARD1 heterodimer, a tumor suppressor complex with ubiquitin E3 ligase activity. Curr Opin Genet Dev 12, 86-91.
4) Baldassarre, G., Battista, S., Belletti, B., Thakur, S., Pentimalli, F., Trapasso, F., Fedele, M., Pierantoni, G., Croce, C. M., and Fusco, A. (2003). Negative regulation of BRCA1 gene expression by HMGA1 proteins accounts for the reduced BRCA1 protein levels in sporadic breast carcinoma. Mol Cell Biol 23, 2225-2238.
5) Beaudenon, S. L., Huacani, M. R., Wang, G., McDonnell, D. P., and Huibregtse, J. M. (1999). Rsp5 ubiquitin-protein ligase mediates DNA damage-induced degradation of the large subunit of RNA polymerase II in Saccharomyces cerevisiae. Mol Cell Biol 19, 6972-6979.
6) Bregman, D. B., Halaban, R., van Gool, A. J., Henning, K. A., Friedberg, E. C., and Warren, S. L. (1996). UV-induced ubiquitination of RNA polymerase II: a novel modification deficient in Cockayne syndrome cells. Proc Natl Acad Sci USA 93, 11586-11590.
7) Briand, J. F., Navarro, F., Rematier, P., Boschiero, C., Labarre, S., Werner, M., Shpakovski, G. V., and Thuriaux, P. (2001). Partners of Rpb8p, a small subunit shared by yeast RNA polymerases I, II and III. Mol Cell Biol 21, 6056-6065.
8) Brzovic, P. S., Keeffe, J. R., Nishikawa, H., Miyamoto, K., Fox, D., 3rd, Fukuda, M., Ohta, T., and Klevit, R. (2003). Binding and recognition in the assembly of an active BRCA1/BARD1 ubiquitin-ligase complex. Proc Natl Acad Sci USA 100, 5646-5651.
9) Catteau, A., and Morris, J. R. (2002). BRCA1 methylation: a significant role in tumour development? Semin Cancer Biol 12, 359-371.
10) Chau, V., Tobias, J. W., Bachmair, A., Marriott, D., Ecker, D. J., Gonda, D. K., and Varshavsky, A. (1989). A multiubiquitin chain is confined to specific lysine in a targeted short-lived protein. Science 243, 1576-1583.
11) Chen, A., Kleiman, F. E., Manley, J. L., Ouchi, T., and Pan, Z. Q. (2002). Autoubiquitination of the BRCA1*BARD1 RING ubiquitin ligase. J Biol Chem 277, 22085-22092.
12) Chiba, N., and Parvin, J. D. (2002). The BRCA1 and BARD1 association with the RNA polymerase II holoenzyme. Cancer Res 62, 4222-4228.
13) Cortez, D., Wang, Y., Qin, J., and Elledge, S. J. (1999). Requirement of ATM-dependent phosphorylation of brca1 in the DNA damage response to double-strand breaks. Science 286, 1162-1166.
14) Cramer, P., Bushnell, D. A., and Kornberg, R. D. (2001). Structural basis of transcription: RNA polymerase II at 2.8 angstrom resolution. Science 292, 1863-1876.
15) Deng, C. X. (2002). Roles of BRCA1 in centrosome duplication. Oncogene 21, 6222-6227.
16) Hashizume, R., Fukuda, M., Maeda, I., Nishikawa, H., Oyake, D., Yabuki, Y., Ogata, H., and Ohta, T. (2001). The RING heterodimer BRCA1-BARD1 is a ubiquitin ligase inactivated by a breast cancer-derived mutation. J Biol Chem 276, 14537-14540.
17) Hershko, A., and Ciechanover, A. (1998). The ubiquitin system. Annu Rev Biochem 67, 425-479.
18) Huibregtse, J. M., Yang, J. C., and Beaudenon, S. L. (1997). The large subunit of RNA polymerase II is a substrate of the Rsp5 ubiquitin-protein ligase. Proc Natl Acad Sci USA 94, 3656-3661.
19) King, M. C., Marks, J. H., and Mandell, J. B. (2003). Breast and ovarian cancer risks due to inherited mutations in BRCA1 and BRCA2. Science 302, 643-646.
20) Kleiman, F. E., Wu-Baer, F., Fonseca, D., Kaneko, S., Baer, R., and Manley, J. L. (2005). BRCA1/BARD1 inhibition of mRNA 3′ processing involves targeted degradation of RNA polymerase II. Genes Dev 19, 1227-1237.
21) Krum, S. A., Miranda, G. A., Lin, C., and Lane, T. F. (2003). BRCA1 associates with processive RNA polymerase II. J Biol Chem 278, 52012-52020.
22) Lane, T. F. (2004). BRCA1 and transcription. Cancer Biol Ther 3, 528-533.
23) Le Page, F., Randrianarison, V., Marot, D., Cabannes, J., Perricaudet, M., Feunteun, J., and Sarasin, A. (2000). BRCA1 and BRCA2 are necessary for the transcription-coupled repair of the oxidative 8-oxoguanine lesion in human cells. Cancer Res 60, 5548-5552.
24) Lee, K. B., Wang, D., Lippard, S. J., and Sharp, P. A. (2002). Transcription-coupled and DNA damage-dependent ubiquitination of RNA polymerase II in vitro. Proc Natl Acad Sci USA 99, 4239-4244.
25) Ljungman, M., and Zhang, F. (1996). Blockage of RNA polymerase as a possible trigger for u.v. light-induced apoptosis. Oncogene 13, 823-831.
26) Mallery, D. L., Vandenberg, C. J., and Hiom, K. (2002). Activation of the E3 ligase function of the BRCA1/BARD1 complex by polyubiquitin chains. Embo J 21, 6755-6762.
27) Morris, J. R., and Solomon, E. (2004). BRCA1: BARD1 induces the formation of conjugated ubiquitin structures, dependent on K6 of ubiquitin, in cells during DNA replication and repair. Hum Mol Genet. 13, 807-817.
28) Nishikawa, H., Ooka, S., Sato, K., Arima, K., Okamoto, J., Klevit, R. E., Fukuda, M., and Ohta, T. (2004). Mass spectrometric and mutational analyses reveal Lys-6-linked polyubiquitin chains catalyzed by BRCA1-BARD1 ubiquitin ligase. J Biol Chem 279, 3916-3924.
29) Ohta, T., and Fukuda, M. (2004). Ubiquitin and breast cancer. Oncogene 23, 2079-2088.
30) Ratner, J. N., Balasubramanian, B., Corden, J., Warren, S. L., and Bregman, D. B. (1998). Ultraviolet radiation-induced ubiquitination and proteasomal degradation of the large subunit of RNA polymerase II. Implications for transcription-coupled DNA repair. J Biol Chem 273, 5184-5189.
31) Rockx, D. A., Mason, R., van Hoffen, A., Barton, M. C., Citterio, E., Bregman, D. B., van Zeeland, A. A., Vrieling, H., and Mullenders, L. H. (2000). UV-induced inhibition of transcription involves repression of transcription initiation and phosphorylation of RNA polymerase II. Proc Natl Acad Sci USA 97, 10503-10508.
32) Ruffner, H., Joazeiro, C. A., Hemmati, D., Hunter, T., and Verma, I. M. (2001). Cancer-predisposing mutations within the RING domain of BRCA1: loss of ubiquitin protein ligase activity and protection from radiation hypersensitivity. Proc Natl Acad Sci USA 98, 5134-5139.
33) Sato, K., Hayami, R., Wu, W., Nishikawa, T., Nishikawa, H., Okuda, Y., Ogata, H., Fukuda, M., and Ohta, T. (2004). Nucleophosmin/B23 is a candidate substrate for the BRCA1-BARD1 ubiquitin ligase. J Biol Chem 279, 30919-30922.
34) Scully, R., Anderson, S. F., Chao, D. M., Wei, W., Ye, L., Young, R. A., Livingston, D. M., and Parvin, J. D. (1997a). BRCA1 is a component of the RNA polymerase II holoenzyme. Proc Natl Acad Sci USA 94, 5605-5610.
35) Scully, R., Chen, J., Ochs, R. L., Keegan, K., Hoekstra, M., Feunteun, J., and Livingston, D. M. (1997b). Dynamic changes of BRCA1 subnuclear location and phosphorylation state are initiated by DNA damage. Cell 90, 425-435.
36) Shen, S. X., Weaver, Z., Xu, X., Li, C., Weinstein, M., Chen, L., Guan, X. Y., Ried, T., and Deng, C. X. (1998). A targeted disruption of the murine Brca1 gene causes gamma-irradiation hypersensitivity and genetic instability. Oncogene 17, 3115-3124.
37) Shpakovski, G. V., Acker, J., Wintzerith, M., Lacroix, J. F., Thuriaux, P., and Vigneron, M. (1995). Four subunits that are shared by the three classes of RNA polymerase are functionally interchangeable between Homo sapiens and Saccharomyces cerevisiae. Mol Cell Biol 15, 4702-4710.
38) Starita, L. M., Horwitz, A. A., Keogh, M. C., Ishioka, C., Parvin, J. D., and Chiba, N. (2005). BRCA1/BARD1 ubiquitinate phosphorylated RNA polymerase II. J Biol Chem 280, 24498-24505.
39) Takeda, S., Naruse, S., and Yatani, R. (1967). Effects of ultra-violet microbeam irradiation of various sites of HeLa cells on the synthesis of RNA, DNA and protein. Nature 213, 696-697.
40) Tibbetts, R. S., Cortez, D., Brumbaugh, K. M., Scully, R., Livingston, D., Elledge, S. J., and Abraham, R. T. (2000). Functional interactions between BRCA1 and the checkpoint kinase ATR during genotoxic stress. Genes Dev 14, 2989-3002.
41) van den Boom, V., Jaspers, N. G., and Vermeulen, W. (2002). When machines get stuck—obstructed RNA polymerase II: displacement, degradation or suicide. Bioessays 24, 780-784.
42) Venkitaraman, A. R. (2002). Cancer susceptibility and the functions of BRCA1 and BRCA2. Cell 108, 171-182.
43) White, R. J. (2004). RNA polymerase III transcription and cancer. Oncogene 23, 3208-3216.
44) Wu-Baer, F., Lagrazon, K., Yuan, W., and Baer, R. (2003). The BRCA1/BARD1 heterodimer assembles polyubiquitin chains through an unconventional linkage involving lysine residue K6 of ubiquitin. J Biol Chem 278, 34743-34746.
45) Xu, B., Kim, S. T., Lim, D. S., and Kastan, M. B. (2002a). Two molecularly distinct G(2)/M checkpoints are induced by ionizing irradiation. Mol Cell Biol 22, 1049-1059.
46) Xu, B., O'Donnell, A. H., Kim, S. T., and Kastan, M. B. (2002b). Phosphorylation of serine 1387 in Brca1 is specifically required for the Atm-mediated S-phase checkpoint after ionizing irradiation. Cancer Res 62, 4588-4591.
47) Xu, X., Weaver, Z., Linke, S. P., Li, C., Gotay, J., Wang, X. W., Harris, C. C., Ried, T., and Deng, C. X. (1999). Centrosome amplification and a defective G2-M cell cycle checkpoint induce genetic instability in BRCA1 exon 11 isoform-deficient cells. Mol Cell 3, 389-395.
48) Zheng, L., Li, S., Boyer, T. G., and Lee, W. H. (2000). Lessons learned from BRCA1 and BRCA2. Oncogene 19, 6159-6175.
49) Zhong, Q., Chen, C. F., Li, S., Chen, Y., Wang, C. C., Xiao, J., Chen, P. L., Sharp, Z. D., and Lee, W. H. (1999). Association of BRCA1 with the hRad50-hMre11-p 95 complex and the DNA damage response. Science 285, 747-750.

### INDUSTRIAL APPLICABILITY

The present invention provides a method for ubiquitinating common subunit of RNA polymerases, a method for suppressing ubiquitination, and a method for establishing cells sensitive to DNA damage. The present invention also provides a pharmaceutical composition comprising BRCA1-BARD1. The method of the present invention can be employed in the field of apoptosis and transcriptional control by ubiquitination of RNA polymerase. By controlling RPB8 ubiquitination via BRCA1, sensitivity of cells to DNA damage is changed, which is useful for the treatment of cancer or the like with drugs causative of DNA damage. The pharmaceutical composition of the invention is valuable for cancer treatment.

What is claimed is:

1. A method for ubiquitinating of common subunit of RNA polymerases, comprising bringing the RNA polymerases into contact with BRCA1-BARD1.

2. The method according to claim 1, wherein the common subunit of RNA polymerases is RPB8.

3. A method for suppressing BRCA1-mediated ubiquitination of RPB8, comprising mutating a lysine residue in an amino acid sequence of RPB8.

4. A method for producing a cell sensitive to a DNA-damaging environment, comprising suppressing ubiquitination of RPB8 through mutation of the amino acid sequence of RPB8 in a cell to impair sensitivity to a DNA-damaging environment to the cell.

5. A pharmaceutical composition comprising BRCA1-BARD1 or a gene thereof.

6. A pharmaceutical composition according to claim 5 for treating cancer.
